SEQUENCE LISTING (2 sequences)

SEQ ID NO: 1 - protein, 598 aa, Mus musculus
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPSFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 2 - protein, 598 aa, Mus musculus; identical to SEQ ID NO: 1

(19) United States
(12) Patent Application Publication
(10) Pub. No.: US 2023/0190873 A1
(43) Pub. Date: Jun. 22, 2023

(54) METHODS AND COMPOSITIONS RELATED TO THE TREATMENT OF NURR1- AND PPARy-MEDIATED CONDITIONS

(71) Applicant: The McLean Hospital Corporation, Belmont, MA (US)

(72) Inventors: Kwang-Soo Kim, Lexington, MA (US); Yongwoo Jang, Belmont, MA (US); Chun-Hyung Kim, Lexington, MA (US)

(21) Appl. No.: 17/869,388

(22) Filed: Jul. 20, 2022

Related U.S. Application Data
(63) Continuation of application No. 16/633,741, filed on Jan. 24, 2020, now abandoned.
(60) Provisional application No. 62/537,634, filed on Jul. 27, 2017.

(51) Int. Cl. A61K 38/17; A61K 31/191; A61K 31/4706; A61K 45/06

(52) U.S. Cl. CPC A61K 38/177; A61K 31/191; A61K 31/4706; A61K 45/06

(57) ABSTRACT

Provided herein are methods and compositions for treating a Nurr1-mediated and/or PPARγ-mediated condition. Also provided herein are methods and compositions for increasing Nurr1 or PPARγ activity and/or levels in a cell.

### CROSS REFERENCE TO RELATED APPLICATIONS

This application is a continuation of application of U.S. patent application Ser. No. 16/633,741, filed Jan. 24, 2020, which is a 371 National Phase Entry of International Patent Application No. PCT/US2018/044094, filed on Jul. 27, 2018, which claims benefit under 35 U.S.C. § 119(e) of U.S. Provisional Application No. 62/537,634 filed Jul. 27, 2017, the contents of which are incorporated herein by reference in their entirety.

### GOVERNMENT SUPPORT

This invention was made with government support under Grant No. NS084860 awarded by the National Institutes of Health. The government has certain rights in the invention.

### SEQUENCE LISTING

This application contains a Sequence Listing that has been submitted electronically as an XML file named “04843-0067002_SL_ST26.XML.” The XML file, created on Nov. 15, 2022, is 3,995 bytes in size. The material in the XML file is hereby incorporated by reference in its entirety.

### TECHNICAL FIELD

The technology described herein relates to the diagnosis and treatment of Nurr1- and PPARγ-mediated conditions, e.g, neurological, autoimmune, and immune conditions.

### BACKGROUND

Nurr1 and PPARγ dysfunction, e.g., low activity and/or expression is known to contribute to the development and progression of a number of diseases. Accordingly, therapeutic approaches to upregulating the activity of these transcription factors are potentially beneficial to patients suffering from a variety of conditions.

### SUMMARY

Described herein is the discovery that certain prostaglandins (or analogs thereof) can specifically activate the transcriptional function of Nurr1 and/or PPARγ. This discovery permits novel methods of treating diseases mediated by Nurr-1 and/or PPARγ by administering one or more of these prostaglandins, thereby correcting or reducing the pathological lack of Nurr-1 and/or PPARγ activity.

One aspect of the invention described here within relates to a method of treating a Nurr1-mediated and/or PPARγ-mediated condition in a subject in need thereof, the method comprising administering to the subject at least one of: prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2.

In one embodiment, the Nurr1-mediated and/or PPARγ-mediated condition is selected from the group consisting of: neurodegenerative disorders; an inflammatory disease; Parkinson's disease; Alzheimer's disease; schizophrenia; immune disorders; mild cognitive impairment; restless leg syndrome; and autoimmune disorders.

In one embodiment, the PGE1; PGE2: PGE3; PGH1; PGH2; PGH3; PGF2a; PGA1; PGA2; PGA3; PGB1; PGB2; PGB3; PGJ2; 15-d-PGJ2; and/or carbocyclic TxA2 is administered in combination with chloroquine or a choloroquine derivative and/or the subject is further administered a cholorquine or cholorquine derivative.

In another embodiment, the subject in need thereof is administered at least one of: PGH1; PGH2; PGH3; PGF2a; PGA1; PGA2; PGA3; PGB1; PGB2; PGB3; PGJ2; 15-d-PGJ2; and carbocyclic TxA2.

In one embodiment, the subject is not administered PGE3.

In another embodiment, the subject is not administered PGE1; PGE2; or PGE3.

An aspect of the invention described herein relates to a method of increasing the level and/or activity of Nurr1 and/or PPARγ in a cell, the method comprising contacting the cell with at least one of: PGE1; PGE2: PGE3; PGH1; PGH2; PGH3; PGF2a; PGA1; PGA2; PGA3; PGB1; PGB2; PGB3; PGJ2; 15-d-PGJ2; and/or carbocyclic TxA2

In one embodiment, the level of Nurr1 is the level of Nurr1 mRNA transcript.

An another aspect present herein describes a method of treating a Nurr1-mediated and/or PPARγ-mediated condition in a subject in need thereof, the method comprising administering to the subject a vector comprising a nucleic acid sequence encoding a Nurr1 polypeptide.

In one embodiment, the Nurr1 polypeptide comprises an amino acid substitution at one or more of the following residues: K554, K558, K590, K577, and C566.

In one embodiment, the Nurr1 polypeptide encoded by the vector is transcribed in the subject at the same or higher transcriptional level than the endogenous Nurr1.

In another embodiment, the Nurr1-mediated and/or PPARγ-mediated condition is selected from the group consisting of: neurodegenerative disorders; an inflammatory disease; Parkinson's disease; Alzheimer's disease; Schizophrenia; immune disorders; mild cognitive impairment; restless leg syndrome; and autoimmune disorders.

An aspect of the invention presented herein describes a method of treating a Nurr1-mediated and/or PPARγ-mediated condition in a subject in need thereof, the method comprising administering to the subject at least one of: prostaglandin E (PGE) 1; PGE2: prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2.

In one embodiment, the Nurr1-mediated and/or PPARγ-mediated condition is selected from the group consisting of: neurodegenerative disorders; an inflammatory disease; Parkinson's disease; Alzheimer's disease; schizophrenia; immune disorders; mild cognitive impairment; restless leg syndrome; and autoimmune disorders.

In another embodiment, the PGE1; PGE2: PGH1; PGH2; PGH3; PGF2a; PGA1; PGA2; PGA3; PGB1; PGB2; PGB3; PGJ2; 15-d-PGJ2; and/or carbocyclic TxA2 is administered in combination with chloroquine or a choloroquine derivative.

In yet another embodiment, the subject is further administered PGE3.

Another aspect of the invention described herein relates to a composition comprising at least one prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2, for use in treating a Nurr1-mediated and/or PPARγ-mediated condition in a subject in need thereof.

In one embodiment, the Nurr1-mediated and/or PPARγ-mediated condition is selected from the group consisting of: neurodegenerative disorders; an inflammatory disease; Parkinson's disease; Alzheimer's disease; schizophrenia; immune disorders; mild cognitive impairment; restless leg syndrome; and autoimmune disorders.

In another embodiment, the composition further comprises a chloroquine or a choloroquine derivative.

In another embodiment, the composition comprises at least one of: PGH1; PGH2; PGH3; PGF2a; PGA1; PGA2; PGA3; PGB1; PGB2; PGB3; PGJ2; 15-d-PGJ2; and carbocyclic TxA2.

In one embodiment, the composition does not comprise PGE3.

In another embodiment, the composition does not comprise PGE1; PGE2; or PGE3.

One aspect of the invention relates to a vector comprising a nucleic acid sequence encoding a Nurr1 polypeptide for use in treating a Nurr1-mediated and/or PPARγ-mediated condition in a subject in need thereof.

In one embodiment, the Nurr1 polypeptide comprises an amino acid substitution at one or more of the following residues K554, K558, K590, K577, and C566.

In another embodiment, the Nurr1 polypeptide encoded by the vector is transcribed in the subject at the same or higher transcriptional level than the endogenous Nurr1.

In another embodiment, the Nurr1-mediated and/or PPARγ-mediated condition is selected from the group consisting of: neurodegenerative disorders; an inflammatory disease; Parkinson's disease; Alzheimer's disease; Schizophrenia; immune disorders; mild cognitive impairment; restless leg syndrome; and autoimmune disorders.

Another aspect of the invention described herein relates to a composition comprising at least one of: prostaglandin E (PGE) 1; PGE2: prostaglandin H(PGH)1; PGH2; PGH3; prostaglandin F (PFG)2a; prostaglandin A (PGA)1; PGA2; PGA3; prostaglandin B (PGB)1; PGB2; PGB3; PGD2; 15-d-PGJ2; and/or carbocyclic TxA2, for use in treating a Nurr1-mediated and/or PPARγ-mediated condition in a subject in need thereof.

In one embodiment, the Nurr1-mediated and/or PPARγ-mediated condition is selected from the group consisting of: neurodegenerative disorders; an inflammatory disease; Parkinson's disease; Alzheimer's disease; schizophrenia; immune disorders; mild cognitive impairment; restless leg syndrome; and autoimmune disorders.

In one embodiment, the composition further comprises a chloroquine or a chloroquine derivative.

In another embodiment, the composition further comprises PGE3.

### BRIEF DESCRIPTION OF THE DRAWINGS

FIG. 1 demonstrates that tissue extracts can enhance the transcriptional activation of Nurr1. The graph depicts Nurr1-inducing activities by tissue extracts from brain, lung, heart, and kidney. Tissue extracts highly increased the reporter gene activity indicating Nurr1 LBD-dependent transcription-inducing activity. The effector plasmid pSVGAL4(DBD)Nurr1(LBD) and the reporter gene for p8XUASL-Luc, together with b-galactosidase expression vector, were transiently transfected into neuroblastoma SK-N-BE(2)C cells. 6 h after transfection, cells were treated with the various tissue extracts for 20 h and the reporter activities were determined by luciferase assay and normalized to the b-galactosidase activity. The basal level of transcriptional activity was normalized to 1. The bars represent means +S.E.M. from three independent experiments.

FIGS. 2A-2B depict fractionation and purification of brain or lung extracts to isolate the endogenous ligand.

FIG. 3 depicts a table of the tentative identification of candidates using UPLC-q-TOF-MS analysis.

FIG. 4 depicts table of the tentative identification of candidates using UPLC-q-TOF-MS analysis.

FIG. 5 depicts a table of the tentative identification of candidates using UPLC-q-TOF-MS analysis.

FIG. 6 depicts a graph demonstrating that PGEs increased Nurr1 activity in a dose-dependent manner.

FIG. 7 depicts a schematic of prostaglandin biosynthesis.

FIG. 8 depicts a graph demonstrating that PGEs activate the transcriptional activity of Nurr1 in an LBD-dependent manner.

FIG. 9 depicts a graph demonstrating that PGEs improve Nurr1 function in a cell-specific manner.

FIG. 10 depicts knock-down of Nurr1 LBD.

FIG. 11 demonstrates that PGE and AQ/CQ synergistically increase the Nurr1 function. Graph depicts effects of single or combination treatment with AQ/CQ and prostaglandin (PGE) on Nurr1 activity. As a negative control, human neuroblastoma SK-N-BE(2)C cells transfected with p8XUAS-Luc and pGAL(DBD)Nurr1(LBD) constructs were treated with DMSO alone. As a positive control, cells were treated as described above and harvested 24 h after treatment. An expression vector encoding RSV b-gal was co-transfected to allow normalization for transfection efficiency. Luciferase activity is represented as fold induction after normalization to β-galactosidase compared to cells treated with DMSO alone. Data represent the means ±SEMs of triplicate samples.

FIG. 12 demonstrates that PGE1 and PGE2 activate the transcriptional activity of Nurr1 in a dose-dependent manner.

FIG. 13 depicts the fold induction of reporter constructs of Nurr1-LBD, PPARa-LBD, and PPARγ-LBD by different concentrations of PGH1, PGH2, PGJ2, and 15-d-PGJ2.

FIG. 14 depicts a schematic of the prostaglandin pathway.

FIG. 15 depicts a summary of transcriptional activation (fold increase) of Nurr1-LBD and PPARγ-LBD of various PGs.

FIGS. 16A-16B demonstrate that PGEs and PGHs activate transcriptional function of Nurr1 but not that of PPARγ. FIG. 16A depicts graphs demonstrating that (Left) PGH1 and PGH2 increase transcriptional activity of Nurr1-LBD in a dose dependent manner and that (Right) 15-d-PGJ2 is a well-known endogenous ligand of PPARγ. As expected, 15-d-PGJ2 increases transcriptional activity of PPARγ-LBD. However, PGH1 and PGH2 failed to induce transcriptional activity of PPARγ-LBD. In FIG. 16B (Left) PGE1 and PGE2 increase transcriptional activity of Nurr1-LBD in a dose dependent manner. In FIG. 16(Right) PGE1 and PGE2 failed to induce transcriptional activity of PPARγ-LBD. FIG. 16C depicts a comparison of the half maximal effective concentration (EC50) of PGH2 and PGE2.

FIGS. 17A-17C demonstrate that PGF2a activates transcriptional function of Nurr1 but not that of PPARγ. FIG. 17A depicts the structure of PGF2a. FIG. 17B demonstrates that PGF2a increases transcriptional activity of Nurr1-LBD in a dose dependent manner. FIG. 17C demonstrates that PGF2a failed to induce transcriptional activity of PPARγ-LBD.

FIGS. 18A-18D demonstrate that carbocyclic thromboxane A2 (TxA2) activates transcriptional function of Nurr1 and PPARγ in a dose-dependent manner. In contrast, TxB2 does not activate transcriptional function of Nurr1 and PPARγ. FIG. 18A depicts the structure of TxA2, TxB2, and carbocyclic TxA2 (stable analog of TxA2). FIG. 18B demonstrates that carbocyclic TxA2 increases transcriptional activity of Nurr1-LBD in a dose dependent manner, whereas TxB2 failed to induce transcriptional activity of Nurr1-LBD. FIG. 18C demonstrates that carbocyclic TxA2 increases transcriptional activity of PPARγ-LBD in a dose dependent manner, whereas TxB2 failed to induce transcriptional activity of PPARγ-LBD. FIG. 18D demonstrates that the activity of carbocyclic TxA2 on PPARγ-LBD has more potent than endogenous ligand, 15-d-PGJ2.

FIGS. 19A-19C demonstrate that PGAs and PGBs activate transcriptional function of Nurr1 in a dose-dependent manner. FIG. 19A depicts the structure of PGA2 and PGB2. PGA2 is converted into PGB2 via PGC2. FIG. 19B demonstrates that PGAs (PGA1, PGA2, PGA3) and PGBs (PGB1, PGB2, PGB3) increases transcriptional activity of Nurr1-LBD in a dose dependent manner. FIG. 19C depicts a comparison of the EC50 of PGAs and PGBs. PGBs show 2 times more potent than PGAs.

FIG. 20 demonstrates that PGB1 and PGB2 activate transcriptional function of PPARγ in a dose-dependent manner.

FIG. 21 demonstrates the protective effect of PGE and PGA on primary cultured mDA neurons. MPP+exposure to mDA neurons decreases the number of TH positive dopaminergic neurons. PGA1 or PGE1 treatment significantly rescued the MPP+-induced loss of TH-positive mDA neurons. *p<0.05, ***p<0.001 (one-way ANOVA, Tukey's post hoc test).

FIGS. 22A-22D demonstrate the protective effect of PGE and PGA on MPTP-induced animal model of PD. FIG. 22A depicts a schematic representation of the administration of PGE1 or PGA1 to MPTP-induced animal model of PD. MPTP-treated mice were pre-administrated with PGE1, PGA1 or saline for 3 days before MPTP injection and post-treated for 5 days after MPTP injection. FIG. 22B demonstrates that motor behaviors (rotarod and pole test) were assessed on day 6 following MPTP administration. PGE1 or PGA1 treatment rescues impaired motor behaviors in latency to fall on a rotating rod (Left) and time to descent on the pole (Right) compared to saline-treated mice. *p<0.05, **p<0.01, ***p<0.001 (one-way ANOVA, Tukey's post hoc test. FIGS. 22C-22D depict immunocytochemical analysis of MPTP-treated mice administrated by PGE1 or PGA1. PGE1 or PGA1-treated mice retain a significant number of TH+neurons in the SN (FIG. 22C) and TH density in the striatum (FIG. 22D), compared to saline-treated control mice. *p<0.05, **p<0.01, ***p<0.001 (one-way ANOVA, Tukey's post hoc test).

FIG. 23A. (Left) Each single mutation on K554, K558, or K590 residues of Nurr1-LBD into arginine (R) residue shows a potentiated basal transcriptional activity comparing to wild-type Nurr1-LBD. Moreover, double (K558R/K590R) or triple (K554R/K558R/K590R) mutations exhibit a synergistic potentiation in Nurr1 basal activity comparing to wild-type Nurr1. (Right) Each single mutation on K554, K558, or K590 residues of Nurr1-LBD into alanine (A) residue shows a potentiated basal transcriptional activity comparing to wild-type Nurr1. Moreover, double (K558A/K590A) or triple (K554A/K558A/K590A) mutations exhibit a synergistic potentiation in Nurr1 basal activity comparing to wild-type Nurr1. FIG. 23B. In terms of K577 residue on Nurr1-LBD, acetylated mimic form (K577Q) of Nurr1-LBD shows a potentiated basal transcriptional activity comparing to wild-type Nurr1-LBD. FIG. 23C. In the C566 residue on Nurr1-LBD, point mutations on C566 residue into several residues (A, D, E, K, R, N, Q) also exhibit a potentiated transcriptional activity of Nurr1 comparing to wild-type Nurr1.

FIG. 24 shows effects of prostaglandin on the dopaminergic differentiation of H9, human embryonic stem cell (hESC). After 15 days of differentiation procedure including floor plate induction (day1˜day6), neural precursor induction (day 6˜day 12), and dopaminergic precursor induction (day 12˜day 15), cells were harvested for RNA extraction and cDNA preparation. The real time PCR was performed using cDNA and the matching primers for FoxA2, Lmx1A, and TH to determine the level of dopaminergic differentiation. Prostaglandin treatment including PGA1, PGB1, and PGE1 showed significantly higher expression level of Lmx1A and TH indicating the enhanced dopaminergic differentiation. Especially, PGE1 showed dramatic increase of TH expression in 0.01 mM concentration, which was even stronger than cAMP, well-known dopaminergic inducer molecule (*; p<0.05 compared to vehicle group).

FIG. 25 show titrations of prostaglandin A1 (PGA1), A2 (PGA2), E1 (PGE1), E2 (PGE2) in LPS-induced immunoactivated mouse bone marrow-derived macrophages. When different concentrations were treated in LPS- or poly(I:C)-induced immune-activated mBMMs for 18 hrs, PGA1, PGA2, PGE1 and PGE2 induced suppression of pro-inflammatory genes including TNFα, IL-1β, iNOS and IL-6 in a dose-dependent manner. The maximal effective concentration was 20 μM or 10 μg/ml.

### DETAILED DESCRIPTION

As described herein, the inventors have discovered that certain prostaglandins can activate Nurr1 and/or PPARγ. Notably, this activity is specific to certain prostaglandins, e.g., PGD molecules failed to display activity in the assays described herein. Accordingly, the prostaglandins described herein are contemplated for increasing the level of Nurr1 and/or PPARγ, an approach which has therapeutic implications for a number of Nurr1- and/or PPARγ-mediated conditions. Additionally, synergy of prostaglandins and cholorquinine (or derivatives thereof), are demonstrated herein.

In some aspects of any of the embodiments, described herein is a method of treating a Nurr1-mediated and/or PPARγ-mediated condition in a subject in need thereof, the method comprising administering to the subject at least one of prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2.

In some embodiments of any of the aspects, described herein is a method of treating a Nurr1-mediated and/or PPARγ-mediated condition in a subject in need thereof, the method comprising administering to the subject at least one of prostaglandin B (PGB)1; PGB2; and/or carbocyclic TxA2.

In some embodiments of any of the aspects, described herein is a method of treating a Nurr1-mediated condition in a subject in need thereof, the method comprising administering to the subject at least one of prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; and/or carbocyclic TxA2.

In some embodiments of any of the aspects, described herein is a method of treating a PPARγ-mediated condition in a subject in need thereof, the method comprising administering to the subject at least one of prostaglandin B (PGB)1; PGB2; PGJ2; 15-d-PGJ2; and/or carbocyclic TxA2.

A Nurr1-mediated condition can be a condition known to be associated with or arising from aberrant expression, function, and/or mutations in Nurr1. Non-limiting examples of Nurr1-mediated conditions can include neurodegenerative disorders; an inflammatory disease; Parkinson's disease; Alzheimer's disease; schizophrenia; immune disorders; mild cognitive impairment; restless leg syndrome; and autoimmune disorders.

As used herein, “PPARγ”, “Peroxisome proliferator-activated receptor gamma”, or “PPARG” refers to a PPAR nuclear receptor that forms heterodimers with RXRs to regulate, e.g., expression of genes involved with glucose metabolism, lipid uptake, and adipogenesis. PPARγ can bind with a number of agents, including various polyunsaturated fatty acids like arachidonic acid, members of the 5-Hydroxyicosatetraenoic acid and 5-oxo-eicosatetraenoic acid family, e.g. 5-oxo-15(S)-HETE and 5-oxo-ETE or 15-Hydroxyicosatetraenoic acid family including 15(S)-HETE, 15(R)-HETE, and 15(S)-HpETE. The sequence of PPARγ is known for a number of species, e.g., human PPARγ (NCBI Gene ID: 5468) mRNA (NCBI Ref Seq: NM_001330615.1, NM_015869.4, NM_138711.3, NM_138712.3, and NM_005037.5) and polypeptide (NCBI Ref Seq: NP_001317544.1, NP_056953.2, NP_619725.2, NP_619726.2 and NP_005028.4) sequences.

A PPARγ-mediated condition can be a condition known to be associated with or arising from aberrant expression, function, and/or mutations in PPARγ. Non-limiting examples of PPARγ-mediated conditions can include neurodegenerative disorders; an inflammatory disease; Parkinson's disease; Alzheimer's disease; schizophrenia; immune disorders; mild cognitive impairment; restless leg syndrome; and autoimmune disorders.

As described herein, a number of prostaglandins have been discovered to activate the transcriptional activity of Nurr1 and/or PPARγ and can be administered to subjects to treat the conditions described herein. As used herein “prostaglandin” refers to a class of eicosnoid lipid compounds enzymatically derived from fatty acids, comprising 20 carbon atoms, including a 5 carbon ring. Prostaglandins are also a subclass of the prostanoid class of fatty acid derivatives. Prostaglandins are named with a letter, which designates the ring structure, and number, which indicates the number of double bonds.

In some embodiments of any of the aspects, a prostaglandin administered according to the methods described herein can be a prostaglandin A, e.g., a prostaglandin in which the cyclopentane nucleus comprises substitutions according to Formula XV.

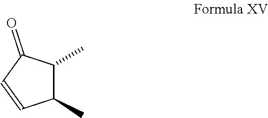

Non-limiting examples of prostaglandin A′s include PGA1, PGA2, and PGA3.

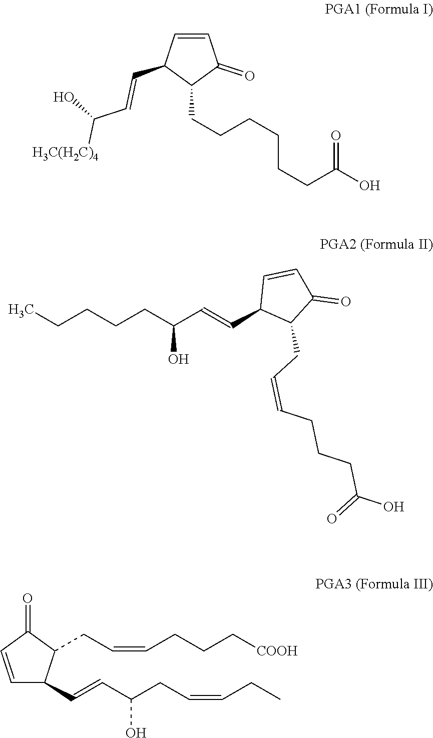

In some embodiments of any of the aspects, a prostaglandin administered according to the methods described herein can be a prostaglandin B, e.g., a prostaglandin in which the cyclopentane nucleus comprises substitutions according to Formula XVI.

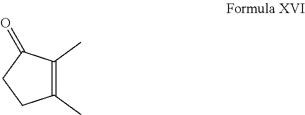

Non-limiting examples of prostaglandin B′s include PGB1, PGB2, and PGB3.

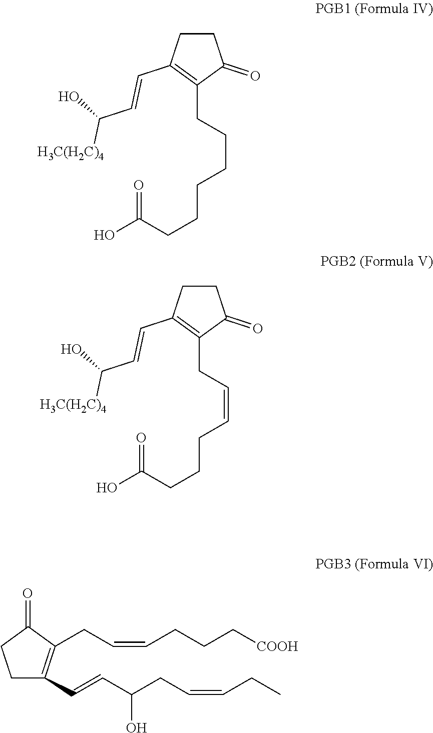

In some embodiments of any of the aspects, a prostaglandin administered according to the methods described herein can be a prostaglandin E, e.g., a prostaglandin in which the cyclopentane nucleus comprises substitutions according to Formula XVII.

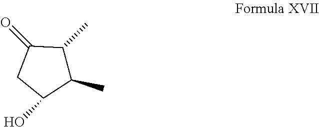

Non-limiting examples of prostaglandin E′s include PGE1, PGE2, and PGE3.

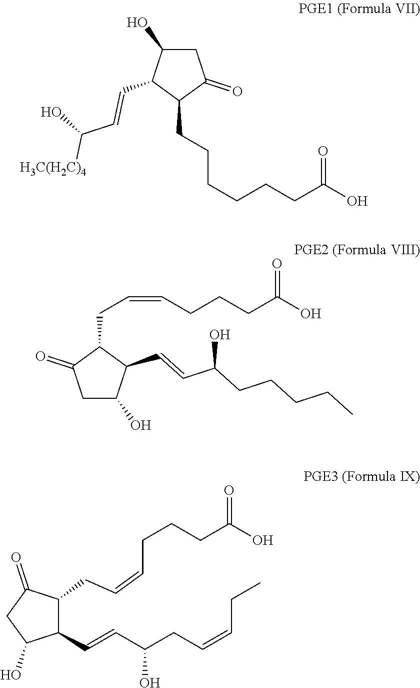

In some embodiments of any of the aspects, a prostaglandin administered according to the methods described herein can be a prostaglandin H, e.g., a prostaglandin in which the cyclopentane nucleus comprises substitutions according to Formula XVIII.

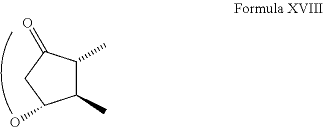

Non-limiting examples of prostaglandin H′s include PGH1, PGH2, and PGH3.

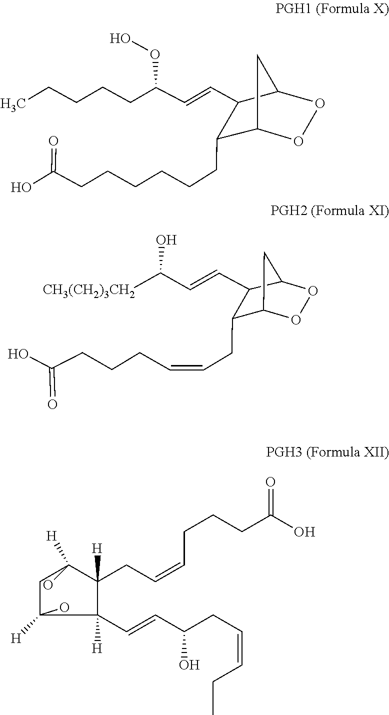

In some embodiments of any of the aspects, a prostaglandin administered according to the methods described herein can be a prostaglandin F, e.g., a prostaglandin in which the cyclopentane nucleus comprises substitutions according to Formula XIX.

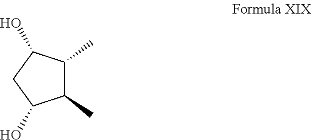

Non-limiting examples of prostaglandin F′s include PGF2a.

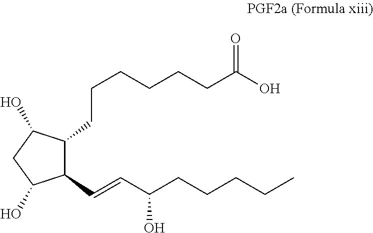 [SMILES: CCCCC[C@H](O)/C=C/[C@@H]1[C@@H](CCCCCCC(=O)O)[C@@H](O)C[C@H]1O]

In some embodiments of any of the aspects, a prostaglandin administered according to the methods described herein can be a prostaglandin J, e.g., a prostaglandin in which the cyclopentane nucleus comprises substitutions according to Formula XXXI.

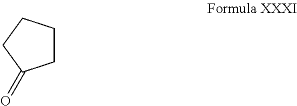

Non-limiting examples of prostaglandin J′s include PGJ2 and 15-d-PGJ2.

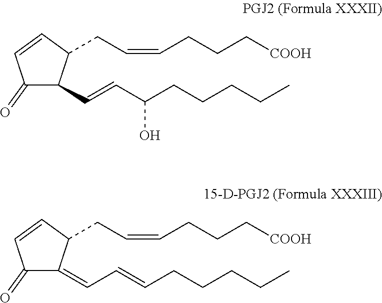 [SMILES: CCCCC[C@H](O)/C=C/[C@H]1C(=O)C=C[C@@H]1C/C=C\CCCC(=O)O ; CCCCC/C=C/C=C1/C(=O)C=C[C@@H]1C/C=C\CCCC(=O)O]

In some embodiments of any of the aspects, carbocylic TxA2 (e.g., a compound of the structure of Formula XIV), can be administered instead of or in addition to one or more prostaglandins.

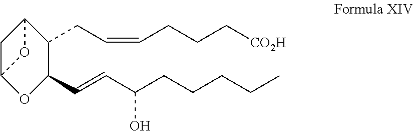 [SMILES: CCCCC[C@H](O)/C=C/[C@H]1O[C@H]2C[C@H](O2)[C@@H]1C/C=C\CCCC(=O)O]

In some embodiments of any of the aspects, multiple prostaglandins and/or carbocylic TxA2 can be administered to the subject, e.g, at the same time, in the same compositions, or at different times or in different compositions. Any composition of the prostaglandins described herein and carbocylic TxA2 are contemplated. As an illustrative example, Table 1 depicts pair-wise combinations that are specifically contemplated herein. The methods described herein are not limited to pairwise combinations and can include 3, 4, 5, 6 or more of the prostaglandins and carbocylic TxA2.

TABLE 1
Suitable pairwise combination are marked with “X”.

| | PGE1 | PGE2 | PGE3 | PGH1 | PGH2 | PGH3 | PGF2a | PGA1 | PGA2 |
|---|---|---|---|---|---|---|---|---|---|
| PGE1 | | X | X | X | X | X | X | X | X |
| PGE2 | X | | X | X | X | X | X | X | X |
| PGE3 | X | X | | X | X | X | X | X | X |
| PGH1 | X | X | X | | X | X | X | X | X |
| PGH2 | X | X | X | X | | X | X | X | X |
| PGH3 | X | X | X | X | X | | X | X | X |
| PGF2a | X | X | X | X | X | X | | X | X |
| PGA1 | X | X | X | X | X | X | X | | X |
| PGA2 | X | X | X | X | X | X | X | X | |
| PGA3 | X | X | X | X | X | X | X | X | X |
| PGB1 | X | X | X | X | X | X | X | X | X |
| PGB2 | X | X | X | X | X | X | X | X | X |
| PGB3 | X | X | X | X | X | X | X | X | X |
| PGJ2 | X | X | X | X | X | X | X | X | X |
| 15-d- | X | X | X | X | X | X | X | X | X |
| PGJ2 | | | | | | | | | |
| Carbocylic | X | X | X | X | X | X | X | X | X |
| TxA2 | | | | | | | | | |

| | | | | | | 15- | Carbocylic |
|---|---|---|---|---|---|---|---|
| | PGA3 | PGB1 | PGB2 | PGB3 | PGJ2 | dPGJ2 | TxA2 |
| PGE1 | X | X | X | X | X | X | X |
| PGE2 | X | X | X | X | X | X | X |
| PGE3 | X | X | X | X | X | X | X |
| PGH1 | X | X | X | X | X | X | X |
| PGH2 | X | X | X | X | X | X | X |
| PGH3 | X | X | X | X | X | X | X |
| PGF2a | X | X | X | X | X | X | X |
| PGA1 | X | X | X | X | X | X | X |
| PGA2 | X | X | X | X | X | X | X |
| PGA3 | | X | X | X | X | X | X |
| PGB1 | X | | X | X | X | X | X |
| PGB2 | X | X | | X | X | X | X |
| PGB3 | X | X | X | | X | X | X |
| PGJ2 | X | X | X | X | | X | X |
| 15-d- | X | X | X | X | X | | X |
| PGJ2 | | | | | | | |
| Carbocylic | X | X | X | X | X | X | |
| TxA2 | | | | | | | |

prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2.

In some embodiments of any of the aspects, the subject is not administered PGE3. In some embodiments of any of the aspects, the subject is not administered PGE1, PGE2, and/or PGE3. In some embodiments of any of the aspects, the subject is not administered a prostaglandin E.

In some embodiments of any of the aspects, a subject in need of treatment for an inflammatory disease is not administered PGE3. In some embodiments of any of the aspects, a subject in need of treatment for an inflammatory disease is not administered PGE1, PGE2, and/or PGE3.

In some embodiments of any of the aspects, the subject is administered one or more of PGE1; PGE2; PGH1; PGH2; PGH3; PGF2a; PGA1; PGA2; PGA3; PGB1; PGB2; PGB3; PGJ2; 15-d-PGJ2; and carbocyclic TxA2. In some embodiments of any of the aspects, the subject is administered one or more of PGH1; PGH2; PGH3; PGF2a; PGA1; PGA2; PGA3; PGB1; PGB2; PGB3; PGJ2; 15-d-PGJ2; and carbocyclic TxA2.

In some embodiments of any of the aspects, a subject in need of treatment for an inflammatory disease is administered one or more of PGE1; PGE2; PGH1; PGH2; PGH3; PGF2a; PGA1; PGA2; PGA3; PGB1; PGB2; PGB3; PGJ2; 15-d-PGJ2; and carbocyclic TxA2. In some embodiments of any of the aspects, a subject in need of treatment for an inflammatory disease is administered one or more of PGH1; PGH2; PGH3; PGF2a; PGA1; PGA2; PGA3; PGB1; PGB2; PGB3; PGJ2; 15-d-PGJ2; and carbocyclic TxA2.

In some embodiments of any of the aspects, a subject treated according to the methods described herein is not in need of treatment for inflammation and/or is not in need of treatment for an inflammatory disease.

In one aspect of any of the embodiments, described herein is a method of increasing the level and/or activity of Nurr1 or PPARγ in a cell, the method comprising contacting the cell with at least one of: PGE1; PGE2: PGE3; PGH1; PGH2; PGH3; PGF2a; PGA1; PGA2; PGA3; PGB1; PGB2; PGB3; PGJ2; 15-d-PGJ2; and/or carbocyclic TxA2. Method for measuring the level and/or activity of Nurr1 or PPARγ are known in the art. For example, methods for measuring the level of a given mRNA and/or polypeptide are known to one of skill in the art, e.g. RT-PCR with primers can be used to determine the level of RNA and Western blotting with an antibody can be used to determine the level of a polypeptide. Nurr1 and/or PPARγ activity can be measuring according to the methods described elsewhere herein, or, e.g., by measuring PPARγ-mediated transcription, e.g, with commercially available kits (e.g., PPARG Transcription Factor Activity Kit ab133101; Abcam; Cambridge, UK).

In some embodiments of any of the aspects, the level of Nurr1 is the level of Nurr1 mRNA transcript. In some embodiments of any of the aspects, the level of PPARγ is the level of PPARγ mRNA transcript.

In one aspect of any of the embodiments, described herein is a method of treating a Nurr1-mediated and/or PPARγ-mediated condition in a subject in need thereof, the method comprising administering to the subject a vector comprising a nucleic acid sequence encoding a Nurr1 polypeptide.

In some embodiments of any of the aspects, the Nurr1 polypeptide can be a full-length wild-type Nurr1 polypeptide, e.g., the sequence of SEQ ID NO 1 or 2. In some embodiments of any of the aspects, the Nurr1 polypeptide can be a full-length wild-type human Nurr1 polypeptide, e.g., the sequence of SEQ ID NO 1 or 2. As used herein, “Nurr1” or “Nuclear receptor related 1 protein” refers to a nuclear receptor that regulates the dopaminergic system. Sequences for Nurr1 are known for a number of species, e.g., human Nurr1 (NCBI Gene ID: 18227) mRNA (NCBI Ref Seq: NM_001139509.1 and NM_013613.2) and polypeptide (NCBI Ref Seq: NP_001132981.1 and NP_038641.1). In some embodiments of any of the aspects, the Nurr1 polypeptide can be an ortholog, variant, and/or allele of SEQ ID NO: 1 or 2.

In some embodiments of any of the aspects, the Nurr1 polypeptide can comprise one or more amino acid substitutions. In some embodiments of any of the aspects, the Nurr1 polypeptide can comprise an amino acid substitution at one or more residues corresponding to K554, K558, K590, K577, and C566 of SEQ ID NO: 1 or 2. In some embodiments of any of the aspects, a substitution comprises substitution to an A, D, E, K, R, N, or Q residue. In some embodiments of any of the aspects, a substitution comprises K554R, K558R, K590R, K577Q, C566A, C566D, C566E, C556K, C566R, C566N, and/or C566Q and combinations thereof. In some embodiments of any of the aspects, the substitutions comprise K558R/K590R or K554R/K558R/K590R. In some embodiments of any of the aspects, the substitutions comprise substitutions at a) K558 and K590 orb) K554, K558, and K590.

In some embodiments of any of the aspects, the vector comprising a nucleic acid encoding a Nurr1 polypeptide is administered in a configuration and/or at a dose such that the Nurr1 polypeptide encoded by the vector is transcribed in the subject at a level which is comparable or higher transcriptional activity than the endogenous Nurr1. This result can be obtained, e.g., by administering a suitable dose of the vector, providing a vector with a promoter as active or more active than the endogenous Nurr1 promoter, providing a vector with multiple copies of a Nurr1-encoding sequence, and/or combinations of the foregoing.

In some embodiments of any of the aspects, a subject treated according to the methods described herein is further administered chloroquine or a choloroquine derivative. Choloroquinine derivatives are known in the art. Exemplary chloroquinine derivatives are described, e.g., in US Patent Publication 2015/0023930A1, which is incorporated by reference herein in its entirety. By way of non-limiting example, in some embodiments of any of the aspects, a chloroquinine derivative can be a compound selected from the group consisting of Formula (XX)-Formula (XXIX):

Formula (XX):

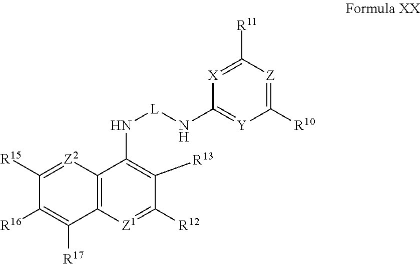

wherein:
X, Y, and Z are independently CR¹⁸, N, O, or S, provided that two of X, Y, and Z are N;
one of Z¹ and Z² is N and the other is CR¹⁴;
R and R¹⁰-R¹⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted; and
L is a linker

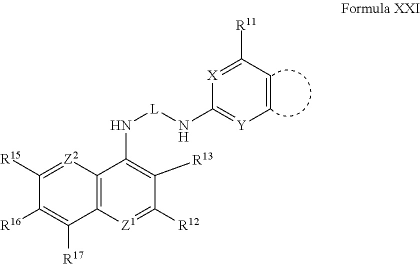

Formula (XXI):
wherein:
X and Y are independently CR¹⁸, N, O, or S;
A and B are independently CR¹⁸, N, O, or S;
one of Z¹ and Z² is N and the other is CR¹⁴;
R and R¹⁰-R¹⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted;
[symbol] forms a cyclyl, heterocyclyl, aryl, or heteroaryl, each of which can be optionally substituted; and
L is a linker;

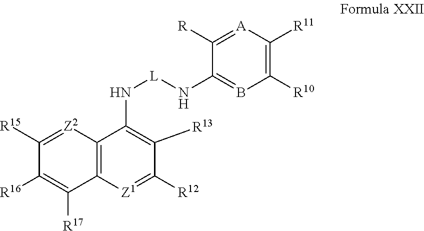

Formula (XXII):
wherein:
A and B are independently CR¹⁸ or N;
one of Z¹ and Z² is N and the other is CR¹⁴;
R and R¹⁰-R¹⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted; and
L is a linker;

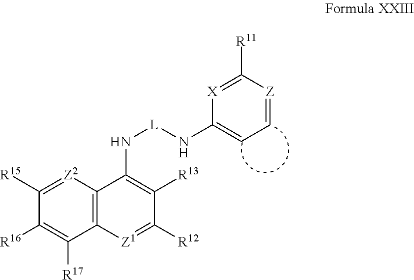

Formula (XXIII):
wherein:
X and Z are independently CR¹⁸, N, O, or S;
one of Z¹ and Z² is N and the other is CR¹⁴;
R and R¹¹-R¹⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted;
[symbol] forms a cyclyl, heterocyclyl, aryl, or heteroaryl, each of which can be optionally substituted; and
L is a linker;

Formula (XXIV):

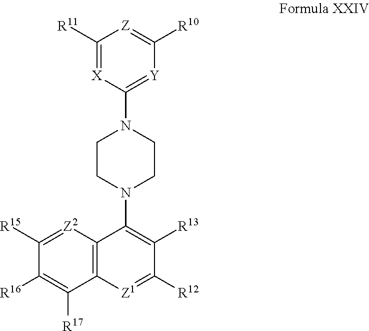

wherein:
X, Y, and Z are independently CR¹⁸, N, O, or S, provided that X and Y are N or Y and Z are N;
one of Z¹ and Z² is N and the other is CR¹⁴;
R and R¹¹-R¹⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted; and
R¹⁰ is linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted;

Formula (XXV):

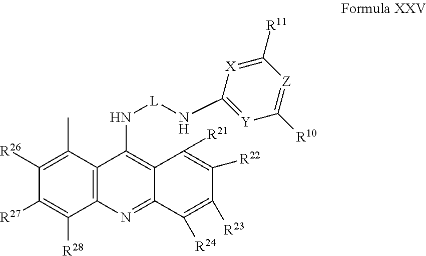

wherein:
X, Y, and Z are independently CR¹⁸, N, O, or S;
R¹⁰, R¹¹ and R²¹-R²⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted; and
L is a linker;

Formula (XXVI):

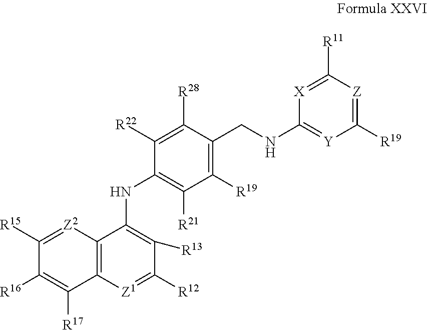

wherein:
X, Y, and Z are independently CR¹⁸, N, O, or S;
one of Z¹ and Z² is N and the other is CR¹⁴; and
R and R¹⁰-R²² are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted.

Formula (XXVII):

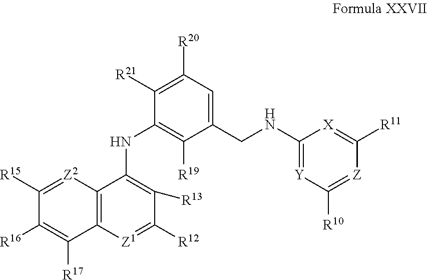

wherein:
X, Y, and Z are independently CR¹⁸, N, O, or S; one of Z¹ and Z² is N and the other is CR¹⁴; and
R and R¹⁰-R²¹ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted;

Formula (XXVIII):

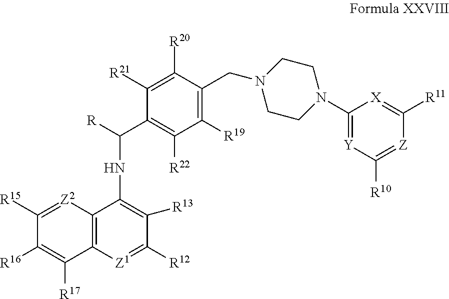

wherein:
X, Y, and Z are independently CR¹⁸, N, O, or S;
one of Z′ and Z² is N and the other is CR¹⁴; and
R and R¹⁰-R²² are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted;

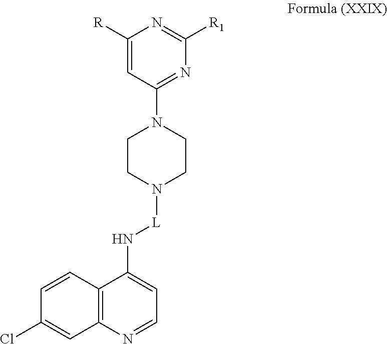

Formula (XXIX):
wherein:
R is hydrogen, CH₃, Cl, CF₃ or CN;
R₁ is cyclic or acylic amine; and
L is an optionally substituted C₂-C₃₀ alkyl, optionally substituted aromatic ring, optionally substituted heteroaryl ring, saturated cyclic ring; amino acid, or peptide;
   and any pharmaceutically acceptable salt, hydrate, solvate, ester, stereoisomer mixture, or enantiomer thereof.

In some embodiments of any of the aspects, a chloroquinine derivative can be a compound of Formula (XXX):

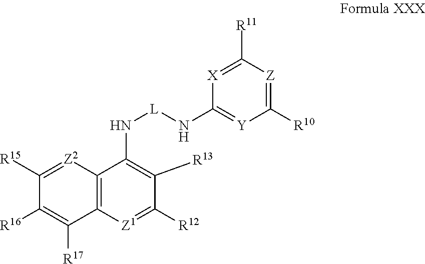

wherein:
X, Y, and Z are independently CR¹⁸, N, O, or S, provided that at least one of X, Y, and Z is N;
Z¹ is CR¹⁴;
Z² is N;
R and R¹⁰-R¹⁸ are independently H, linear or branched alkyl, linear or branched alkenyl, linear or branched alkynyl, cyclyl, heterocyclyl, aryl, heteroaryl, halogen, trifluoromethyl, alkoxy, nitro, cyano, carbonyl, hydroxyl, phenoxy, amino, alkylamino, thiol, or alkylthio, each of which can be optionally substituted; and
L is a linker,
and any pharmaceutically acceptable salt, hydrate, solvate, ester, stereoisomer mixture, or enantiomer thereof.

In some embodiments of any of the aspects, the methods described herein relate to treating a subject having or diagnosed as having a Nurr1- or PPARγ-mediated condition with, e.g., one or more prostaglandins as described herein. Subjects having a Nurr1- or PPARγ-mediated condition, e.g., Alzheimer's disease can be identified by a physician using current methods of diagnosing Alzheimer's disease. Symptoms and/or complications of Alzheimer's disease which characterize these conditions and aid in diagnosis are well known in the art and include but are not limited to, disorientation, cognitive impairment, memory dysfunction, etc. Tests that may aid in a diagnosis of, e.g. Alzheimer's disease include, but are not limited to, the Functional Assessment Staging (FAST) scale, cellular and molecular testing methods disclosed in U.S. Pat. Nos. 7,771,937, 7,595,167, 5,558,0748, and PCT Application No.: WO2009/009457, the content of which is incorporated by reference in its entirety; and protein-based biomarkers for AD, some of which can be detected by non-invasive imaging, e.g., PET, are disclosed in U.S. Pat. No. 7,794,948, the content of which is incorporated by reference in its entirety. A family history of Alzheimer's disease, or exposure to risk factors for Alzheimer's disease can also aid in determining if a subject is likely to have Alzheimer's disease or in making a diagnosis of Alzheimer's disease.

The compositions and methods described herein can be administered to a subject having or diagnosed as having a Nurr1- or PPARγ-mediated condition. In some embodiments of any of the aspects, the methods described herein comprise administering an effective amount of compositions described herein, e.g. a prostaglandin described herein to a subject in order to alleviate a symptom of a Nurr1- or PPARγ-mediated condition. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, or injection administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an active ingredient which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for Nurr1 and/or PPARγ levels and/or activity, among others. The dosage can be determined by a physician and adjusted, as necessary, to suit observed effects of the treatment.

In some embodiments of any of the aspects, the technology described herein relates to a pharmaceutical composition comprising at least one prostaglandin, TxA2, Nurr1 polypeptide, and/or vector encoding a Nurr1 polypeptide as described herein, and optionally a pharmaceutically acceptable carrier. In some embodiments of any of the aspects, the active ingredients of the pharmaceutical composition comprise at least one prostaglandin, TxA2, Nurr1 polypeptide, and/or vector encoding a Nurr1 polypeptide as described herein as described herein. In some embodiments of any of the aspects, the active ingredients of the pharmaceutical composition consist essentially of at least one prostaglandin, TxA2, Nurr1 polypeptide, and/or vector encoding a Nurr1 polypeptide as described herein as described herein. In some embodiments of any of the aspects, the active ingredients of the pharmaceutical composition consist of at least one prostaglandin, TxA2, Nurr1 polypeptide, and/or vector encoding a Nurr1 polypeptide as described herein as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent, as described herein.

In some embodiments of any of the aspects, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of compositions described herein are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the composition described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1 ; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the at least one prostaglandin, TxA2, Nurr1 polypeptide, and/or vector encoding a Nurr1 polypeptide as described herein described herein is administered as a monotherapy, e.g., another treatment for the Nurr1- and/or PPARγ-mediated condition is not administered to the subject.

The methods described herein can further comprise administering a second agent and/or treatment to the subject, e.g. as part of a combinatorial therapy. By way of non-limiting example, if a subject is to be treated for inflammation according to the methods described herein, the subject can also be administered a second agent and/or treatment known to be beneficial for subjects suffering from inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, and the like.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the active ingredient, its potency, and the extent to which symptoms, markers, or indicators of a condition described herein are desired to be reduced, for example the percentage reduction desired for one or more symptoms or markers or the extent to which, for example, markers are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. level or severity of a symptom, or the level and/or activity of Nurr1 and/or PPARγ). It is well within the ability of one skilled in the art to monitor efficacy of administration and/or treatment by measuring any one of such parameters, or any combination of parameters. Efficacy can be assessed in animal models of a condition described herein, for example treatment of a mouse model of a Nurr1- or PPARγ-mediated condition. When using an experimental animal model, efficacy of treatment is evidenced when a statistically significant change in a marker is observed, e.g. the level and/or activity of Nurr1 and/or PPARγ.

In vitro and animal model assays are provided herein which allow the assessment of a given dose of a composition described herein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory disease or condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments of any of the aspects, the inflammatory condition is associated with wound healing. In some embodiments of any of the aspects, the inflammation to be treated according to the methods described herein can be.

A neurodegernative disease is a disease in which nervous cell and/or nervous system function degrades over time, e.g., due to abnormal function, behavior, and/or proliferation in nervous system cells. Non-limiting examples of neurodegenerative diseases can include Alzheimer's disease, Dentatorubropallidoluysian atrophy (DRPLA), Huntington's Disease (HD), Spinocerebellar ataxia Type 1 (SCA1), Spinocerebellar ataxia Type 2 (SCA2), Spinocerebellar ataxia Type 3 (SCA3), Spinocerebellar ataxia 6 (SCA6), Spinocerebellar ataxia Type 7 (SCAT), Spinocerebellar ataxia Type 8 (SCAB), Spinocerebellar ataxia Type 12 (SCA12), Spinocerebellar ataxia Type 17 (SCA17), Spinobulbar Muscular Ataxia/Kennedy Disease (SBMA), Fragile X syndrome (FRAXA), Fragile XE mental retardation (FRAXE), and Myotonic dystrophy (DM).

As used herein, the terms “autoimmune disease” or “autoimmune disorder” refer to a condition that is immune-mediated due to an attack on self-tissues, such as when a subject's own antibodies react with host tissue, but can also involve an immune response to a microorganism.

Autoimmune diseases can include, but are not limited to lupus erythematosus; Wiskott-Aldrich syndrome; autoimmune lymphoproliferative syndrome; myasthenia gravis; rheumatoid arthritis (RA); lupus nephritis; multiple sclerosis; systemic lupus erythematosis; discoid lupus; subacute cutaneous lupus erythematosus; cutaneous lupus erythematosus including chilblain lupus erythematosus; chronic arthritis; Sjogren's syndrome; autoimmune nephritis; autoimmune diabetes nephritis; autoimmune vasculitis; autoimmune hepatitis; autoimmune carditis; autoimmune encephalitis; autoimmune mediated hematological disease, Crohn's disease, ulcerative colitis, vasculitis; ankylosing spondylitis; Behcet's disease; paraneoplastic autoimmunity, dermatomyositis, inflammatory chronic rhinosinusitis, celiac disease, inflammatory bowel disease, Barrett's esophagus, inflammatory gastritis autoimmune nephritis, autoimmune diabetes, Addison's disease, alopecia areata, autoimmune parotitis, dystrophic epidermolysis bullosa, epididymitis, glomerulonephritis, Graves' disease, Guillain-Barr syndrome, Hashimoto's disease, hemolytic anemia, pemphigus vulgaris, psoriasis, rheumatic fever, scleroderma, spondyloarthropathies, thyroiditis, vasculitis, vitiligo, myxedema, pernicious anemia, ulcerative colitis, Goodpasture's syndrome, pemphigus (e.g., pemphigus vulgaris), autoimmune thrombocytopenic purpura, scleroderma with anti-collagen antibodies, mixed connective tissue disease, polymyositis, pernicious anemia, autoimmune-associated infertility, glomerulonephritis (e.g., crescentic glomerulonephritis, proliferative glomerulonephritis), bullous pemphigoid, insulin resistance, and autoimmune diabetes mellitus (type 1 diabetes mellitus; insulin-dependent diabetes mellitus), gastritis, autoimmune hemophilia, and autoimmune uveoretinitis.

In some embodiments of the methods of treating immune conditions as described herein, the subject being administered the compositions described herein has or has been diagnosed with host versus graft disease (HVGD). In a further embodiment, the subject being treated with the methods described herein is an organ or tissue transplant recipient. In other embodiments of the methods of treating immune conditions as described herein, the methods are used for increasing transplantation tolerance in a subject. In some such embodiments, the subject is a recipient of an allogenic transplant. The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

As used herein, the term “immune deficiency” or “immune disease” refers to a condition in which a portion or some portions of cell components constituting an immune system are defective or dysfunction, so that a normal immune mechanism is damaged. In other words, “immune deficiency” means a condition under which: congenital immunity and/or acquired immunity are suppressed and/or decreased. In some embodiments of any of the aspects, the immune-deficiency subject is an immunocompromised subject. Non-limiting examples of immune deficiencies can include AIDS, hypogammaglobulinemia, agammaglobulinemia, granulocyte deficiency, chronic granulomatous disease, asplenia, SCID, complement deficiency, and/or sickle cell anemia.

As used herein, “autoimmune cell” or “autoreactive cell” refers to immune cells that have activity towards and/or recognize cells or biological components of the organism from which the cell is derived. Examples of cells which can be autoimmune cells include, but are not limited to, adult splenocytes, T cells, and B cells. As used herein, the term “immune cell” refers to a cell that is part of the innate and/or adaptive immune systems. Immune cells can be of hematopoietic origin and include, by way of non-limiting example, lymphocytes, B cells, T cells, NK cells, myeloid cells, monocytes, macrophages, eosinophils, mast cells, basophils, granulocytes, dendritic cells, phagocytes, and neutrophils.

As used herein, “mild cognitive impairment” or “MCI”, which is also known as incipient dementia, or isolated memory impairment refers to a brain function syndrome involving the onset and evolution of cognitive impairments beyond those expected based on the age and education of the individual, but which are not significant enough to interfere with their daily activities. It may occur as a transitional stage between normal aging and dementia. At the time the invention was made, prevention and treatments of mild cognitive impairment were not available.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (IV), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments of any of the aspects, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments of any of the aspects, the polypeptide described herein can be a variant of a sequence described herein. In some embodiments of any of the aspects, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, a nucleic acid encoding a polypeptide as described herein (e.g. a Nurr1 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, In some embodiments of any of the aspects, be combined with other suitable compositions and therapies. In some embodiments of any of the aspects, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a condition described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The present invention can be defined in any of the following numbered paragraphs:
1) A method of treating a Nurr1-mediated and/or PPARγ-mediated condition in a subject in need thereof, the method comprising administering to the subject at least one of:
   prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGD2; 15-d-PGJ2; and/or carbocyclic TxA2.
2) The method of paragraph 1, wherein the Nurr1-mediated and/or PPARγ-mediated condition is selected from the group consisting of:
   neurodegenerative disorders; an inflammatory disease; Parkinson's disease; Alzheimer's disease; schizophrenia; immune disorders; mild cognitive impairment; restless leg syndrome; and autoimmune disorders.
3) The method of any of paragraphs 1-2, wherein the PGE1; PGE2: PGE3; PGH1; PGH2; PGH3; PGF2a; PGA1; PGA2; PGA3; PGB1; PGB2; PGB3; PGJ2; 15-d-PGJ2; and/or carbocyclic TxA2 is administered in combination with chloroquine or a choloroquine derivative.
4) The method of any of paragraphs 1-3, wherein the subject is administered at least one of:
   PGE1; PGE2; PGH1; PGH2; PGH3; PGF2a; PGA1; PGA2; PGA3;PGB1; PGB2; PGB3; PGJ2; 15-d-PGJ2; and carbocyclic TxA2.
5) The method of any of paragraphs 1-4, wherein the subject is administered at least one of:
   PGH1; PGH2; PGH3; PGF2a; PGA1; PGA2; PGA3; PGB1; PGB2; PGB3; PGJ2; 15-d-PGJ2; and carbocyclic TxA2.
6) The method of any of paragraphs 1-5, wherein the subject is not administered PGE3.
7) The method of any of paragraphs 1-6, wherein the subject is not administered PGE1; PGE2; or PGE3.
8) A method of increasing the level and/or activity of Nurr1 or PPARγ in a cell, the method comprising contacting the cell with at least one of:
   PGE1; PGE2: PGE3; PGH1; PGH2; PGH3; PGF2a; PGA1; PGA2; PGA3; PGB1; PGB2; PGB3; PGJ2; 15-d-PGJ2; and/or carbocyclic TxA2
9) The method of paragraph 8, wherein the level of Nurr1 is the level of Nurr1 mRNA transcript.
10) A method of treating a Nurr1-mediated and/or PPARγ-mediated condition in a subject in need thereof, the method comprising administering to the subject a vector comprising a nucleic acid sequence encoding a Nurr1 polypeptide.
11) The method of paragraph 10, wherein the Nurr1 polypeptide comprises an amino acid substitution at one or more of the following residues:
   K554, K558, K590, K577, and C566.
12) The method of any of paragraphs 10-11, wherein the Nurr1 polypeptide encoded by the vector is transcribed in the subject at the same or higher transcriptional level than the endogenous Nurr1.
13) The method of any of paragraphs 10-12, wherein the Nurr1-mediated and/or PPARγ-mediated condition is selected from the group consisting of:
   neurodegenerative disorders; an inflammatory disease; Parkinson's disease; Alzheimer's disease; Schizophrenia; immune disorders; mild cognitive impairment; restless leg syndrome; and autoimmune disorders.
14) A method of treating a Nurr1-mediated and/or PPARγ-mediated condition in a subject in need thereof, the method comprising administering to the subject at least one of:
   prostaglandin E (PGE) 1; PGE2: prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2.
15) The method of paragraph 14, wherein the Nurr1-mediated and/or PPARγ-mediated condition is selected from the group consisting of:
   neurodegenerative disorders; an inflammatory disease; Parkinson's disease; Alzheimer's disease; schizophrenia; immune disorders; mild cognitive impairment; restless leg syndrome; and autoimmune disorders.
16) The method of any of paragraphs 14-15, wherein the PGE1; PGE2: PGH1; PGH2; PGH3; PGF2a; PGA1; PGA2; PGA3; PGB1; PGB2; PGB3; PGJ2; 15-d-PGJ2; and/or carbocyclic TxA2 is administered in combination with chloroquine or a choloroquine derivative.
17) The method of any of paragraphs 14-16, wherein the subject is further administered PGE3.
18) A composition comprising at least one of:
   prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2, for use in treating a Nurr1-mediated and/or PPARγ-mediated condition in a subject in need thereof.
19) The composition of paragraph 18, wherein the Nurr1-mediated and/or PPARγ-mediated condition is selected from the group consisting of:
   neurodegenerative disorders; an inflammatory disease; Parkinson's disease; Alzheimer's disease; schizophrenia; immune disorders; mild cognitive impairment; restless leg syndrome; and autoimmune disorders.
20) The composition of any of paragraphs 18-19, further comprising a chloroquine or a choloroquine derivative.
21) The composition of any of paragraphs 18-20, comprising at least one of:
   PGE1; PGE2; PGH1; PGH2; PGH3; PGF2a; PGA1; PGA2; PGA3;PGB1; PGB2; PGB3; PGJ2; 15-d-PGJ2; and carbocyclic TxA2.
22) The composition of any of paragraphs 18-21 comprising at least one of:
   PGH1; PGH2; PGH3; PGF2a; PGA1; PGA2; PGA3; PGB1; PGB2; PGB3; PGJ2; 15-d-PGJ2; and carbocyclic TxA2.
23) The composition of any of paragraphs 18-22, wherein the composition does not comprise PGE3.
24) The composition of any of paragraphs 18-23, wherein the composition does not comprise PGE1; PGE2; or PGE3.
25) A vector comprising a nucleic acid sequence encoding a Nurr1 polypeptide for use in treating a Nurr1-mediated and/or PPARγ-mediated condition in a subject in need thereof.
26) The vector of paragraph 25, wherein the Nurr1 polypeptide comprises an amino acid substitution at one or more of the following residues:
   K554, K558, K590, K577, and C566.
27) The vector of any of paragraphs 25-26, wherein the Nurr1 polypeptide encoded by the vector is transcribed in the subject at the same or higher transcriptional level than the endogenous Nurr1.
28) The vector of any of paragraphs 25-27, wherein the Nurr1-mediated and/or PPARγ-mediated condition is selected from the group consisting of:
   neurodegenerative disorders; an inflammatory disease; Parkinson's disease; Alzheimer's disease; Schizophrenia; immune disorders; mild cognitive impairment; restless leg syndrome; and autoimmune disorders.
29) A composition comprising at least one of:
   prostaglandin E (PGE) 1; PGE2: prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2, for use in treating a Nurr1-mediated and/or PPARγ-mediated condition in a subject in need thereof.
30) The composition of paragraph 29, wherein the Nurr1-mediated and/or PPARγ-mediated condition is selected from the group consisting of:
   neurodegenerative disorders; an inflammatory disease; Parkinson's disease; Alzheimer's disease; schizophrenia; immune disorders; mild cognitive impairment; restless leg syndrome; and autoimmune disorders.
31) The composition of any of paragraphs 29-30, further comprising a chloroquine or a choloroquine derivative.
32) The composition of any of paragraphs 29-31, wherein the composition further comprises PGE3.

The technology described herein is further illustrated by the following examples which in no way should be construed as being further limiting.

### EXAMPLES

### Example 1

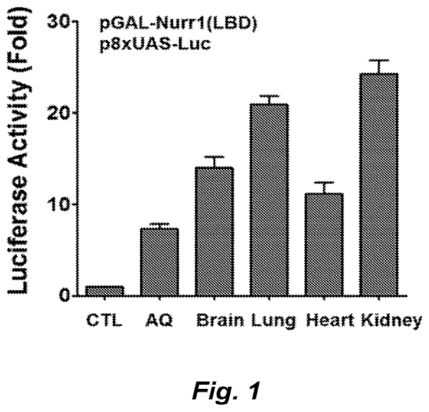

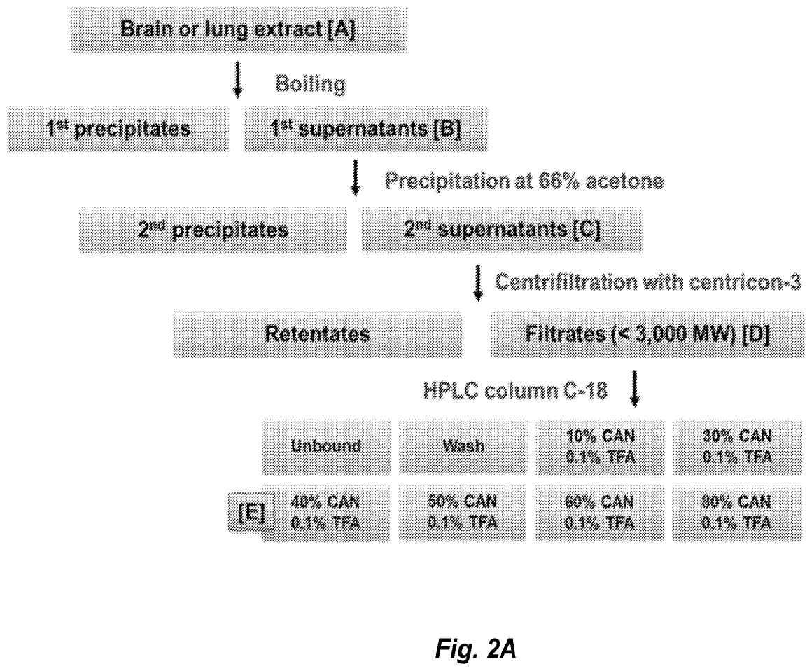

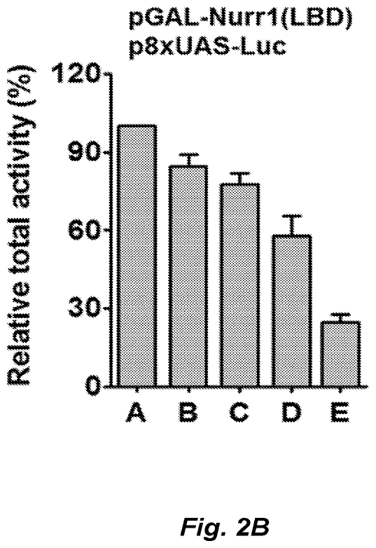

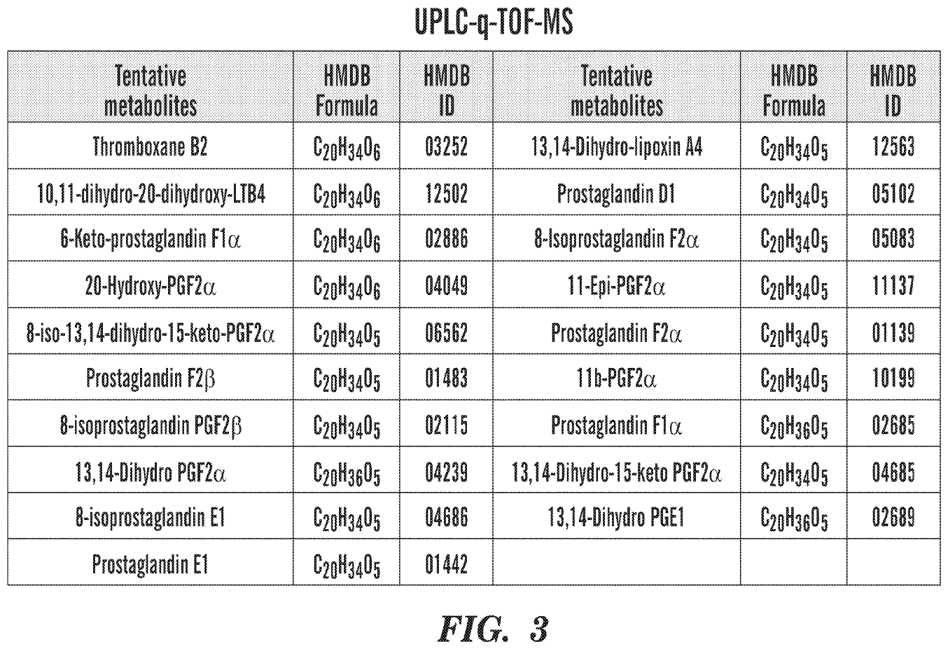

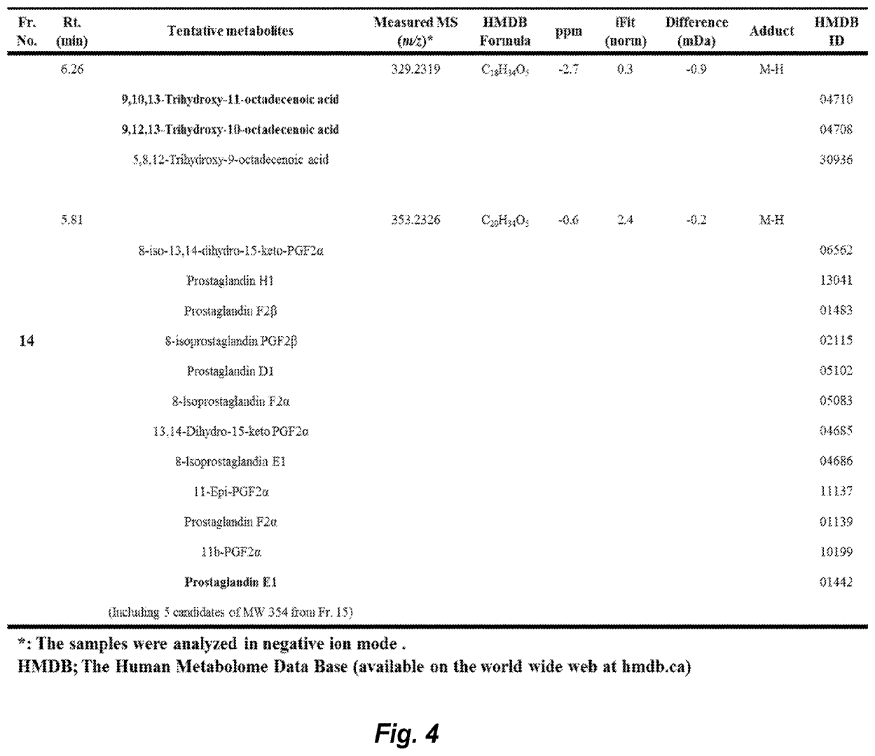

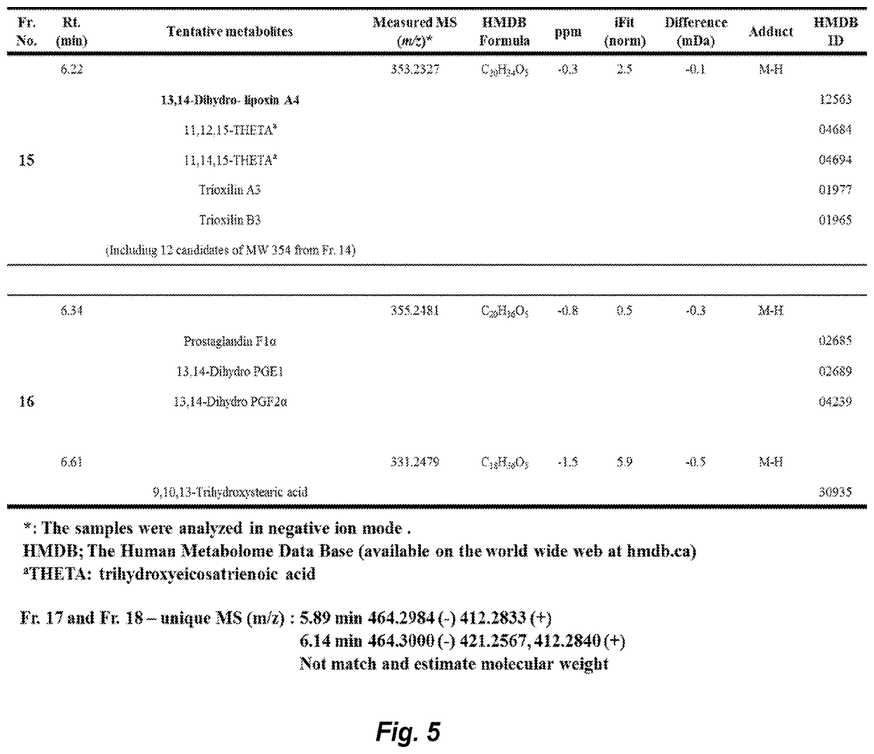

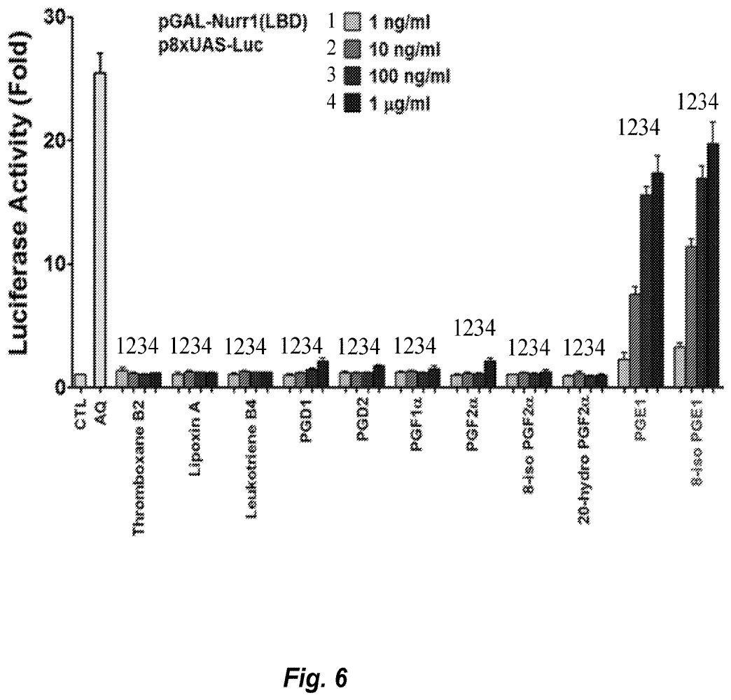

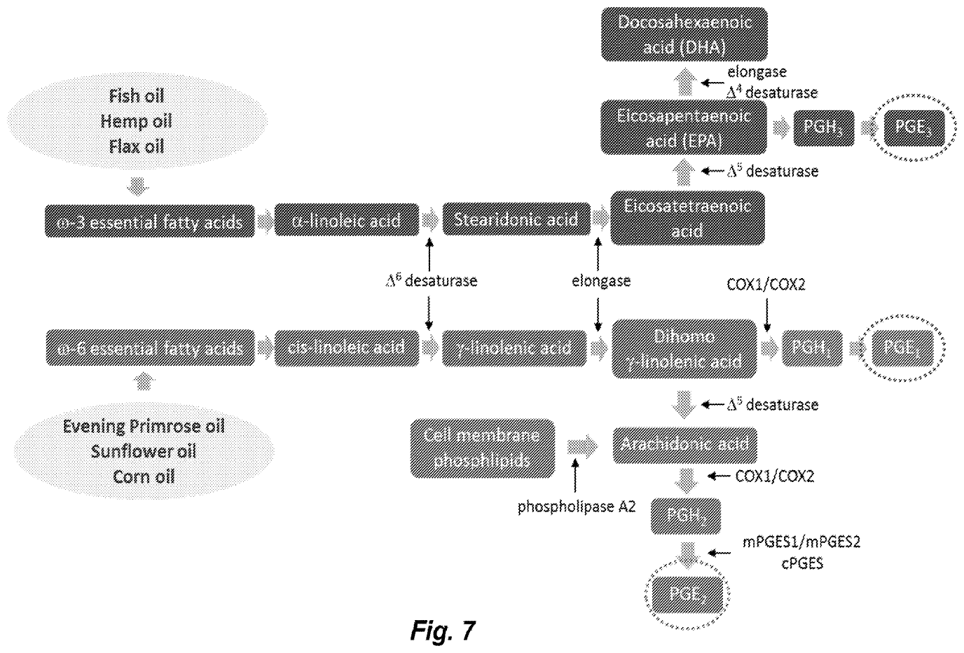

Described herein are methods and kits for inducing the function and activity of Nurr1. Using a high thoughput in vitro assay system, tissue extracts were screened and it was found that there exist significant activities to enhance the transcriptional activities of Nurr1. Using biochemical assays of size-fractionated molecules, an active small molecule fraction of molecular weight between 320 to 370 was identified (FIG. 1, 2A, 2B). Using the UPLC-q-TOF-MS analyses, potential candidate molecules in the fraction were identified and it was found that prostaglandin E (PGE) 1, 2, and 3 specifically and prominently activate the transcription function of Nurr1 (FIGS. 3-5). In contrast, other prostaglandin molecules such as PGD and PGF did not show any detectable activity, showing the specificity of PGE molecules for enhancing the Nurr1 transcriptional activity (FIG. 6). When expression of Nurr1 was down-regulated by its siRNA expression, the activities of PGE molecules were almost completely diminished, showing that their activities are specifically mediated by Nurr1.

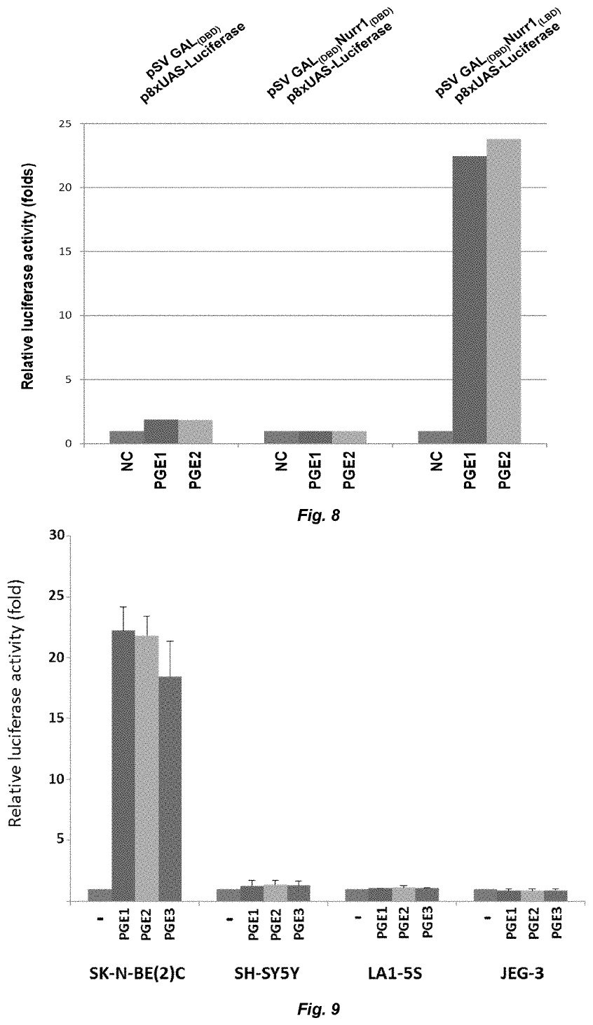

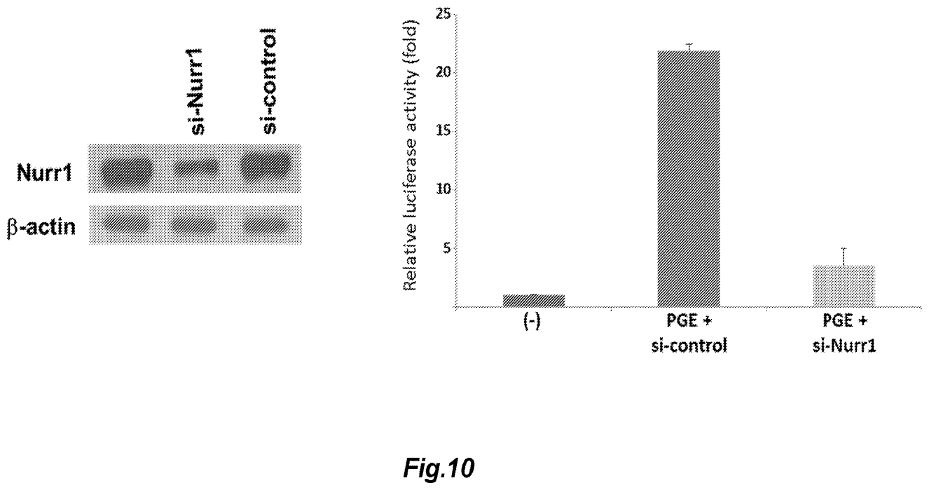

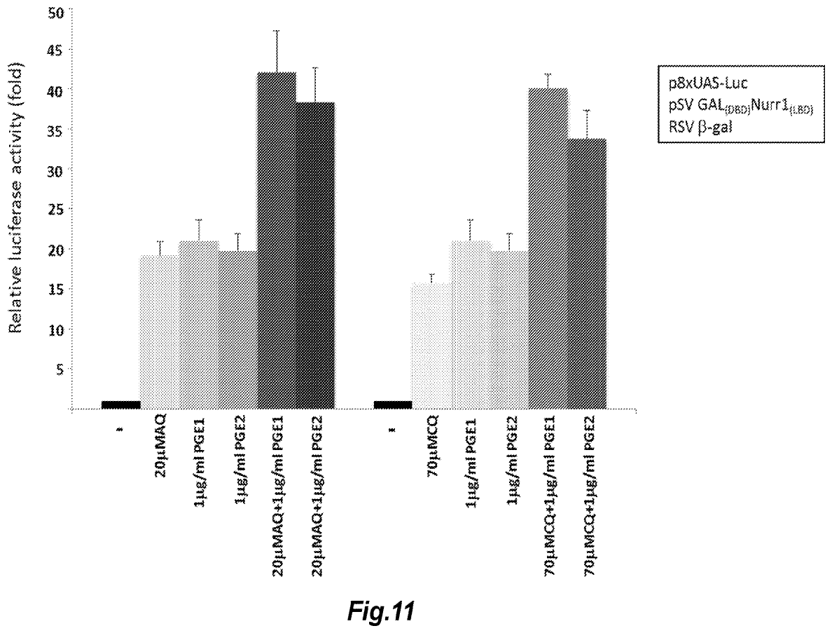

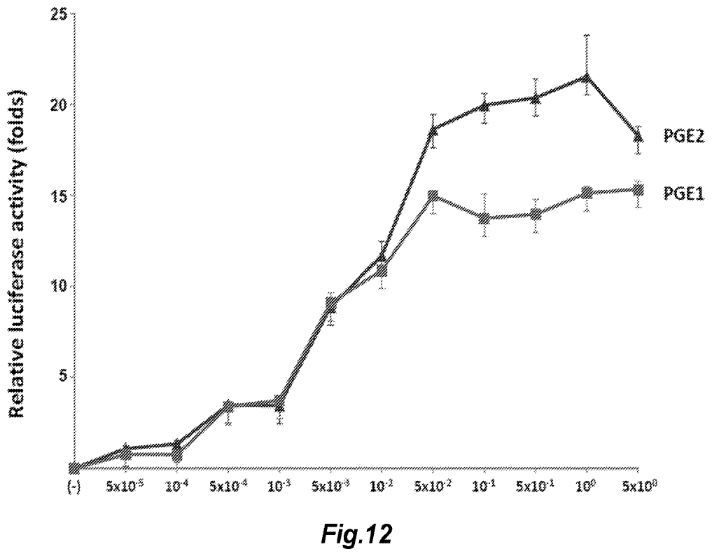

In addition, it was found that PGE molecules activate the Nurr1's function through its ligand binding domain, but not its DNA binding domain, indicating that PGEs (and/or their biological metabolites such as PGA molecules) represent Nurr1's agonists (FIG. 8). Furthermore, PGE molecules can enhance Nurr1's transcriptional repressor function in immune cells, resulting in further repression of proinflammatory gene expression in primary macrophage cells (FIG. 9). Furthermore, PGE molecule can synergistically activate the Nurr1 transcriptional activity together with chloroquine and its derivative (FIG. 11), indicating that the combined treatment of PGE molecule and CQ (or its derivative) can effectively treat human diseases that are related with Nurr1 (e.g., Parkinson's disease, Alzheimer's disease, immunological and autoimmune diseases). This invention permits the efficient treatment of neurodegenerative disorders and immunological/autoimmune diseases in a mechanism-based manner by directly influencing the function of Nurr1 through its ligand binding domain.

Demonstrated herein are:
Identification of PGE1, 2, and 3 (and their biological metabolites) as endogenous activators/agonists of Nurr1 function.
PGE molecules enhance the Nurr1's transcriptional activities for both activator function and repressor function.
PGE molecules show synergistic activation of Nurr1's transcriptional function together with CQ and its derivatives

It is specifically contemplated herein that PGE molecules and derivatives are therapeutic agents for the treatment of neurodegenerative disorders and/or immunological/autoimmune diseases and/or any Nurr1-related diseases by themselves and/or in combination with CQ (or its derivatives).

### Example 2

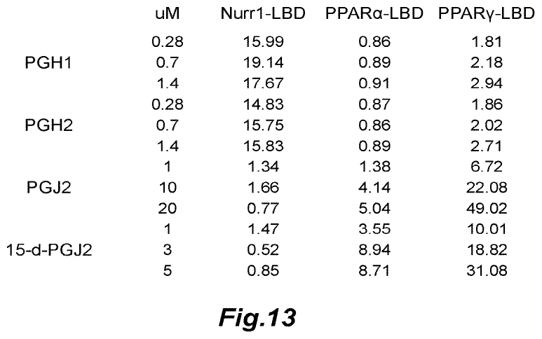

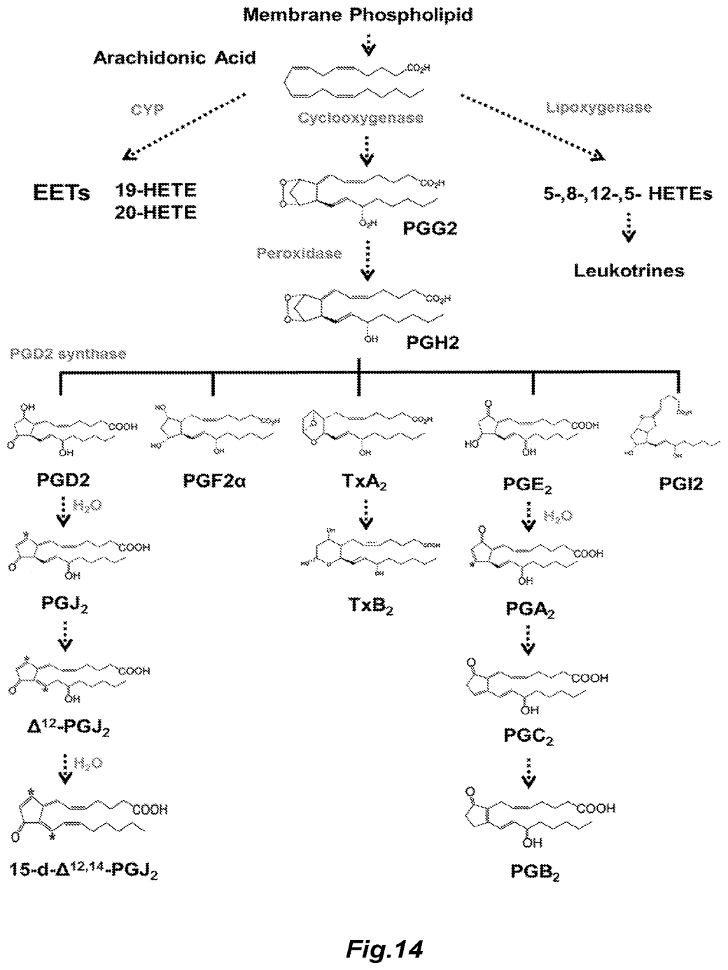

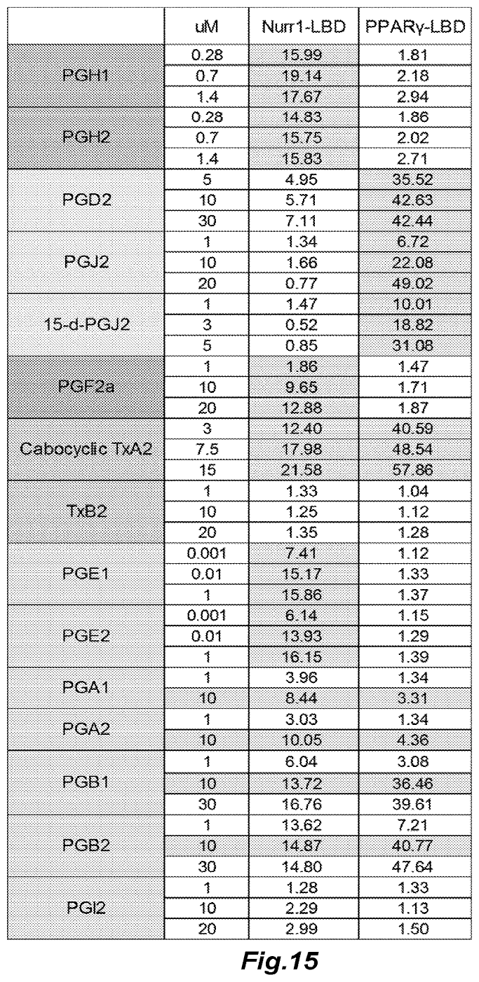

As shown in FIG. 14, PGH1 and 2 are precursors of PGE1 and 2. PGH1 and 2 also robustly activate the transcriptional activities of Nurr1 LBD, but not those of PPARalpha and PPARgamma, indicating specificity (FIG. 13). In contrast, PGJ2 and 15-d-PGJ2 (known ligand of PPARgamma) robustly activated PPARs, not Nurr1 LBD. These results indicate that PGH1, 2, and 3 act as activators of Nurr1 together with PGE1, 2, and 3.

### Example 3

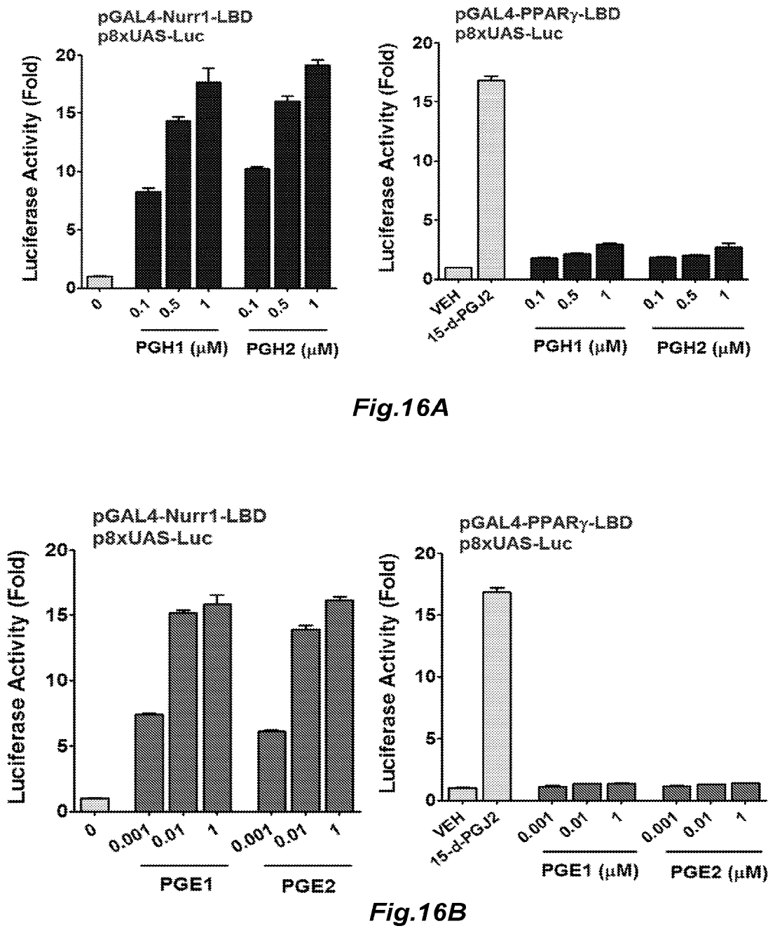

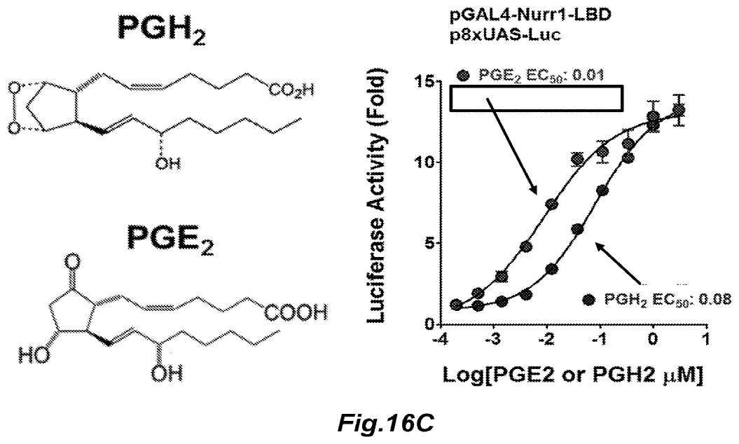

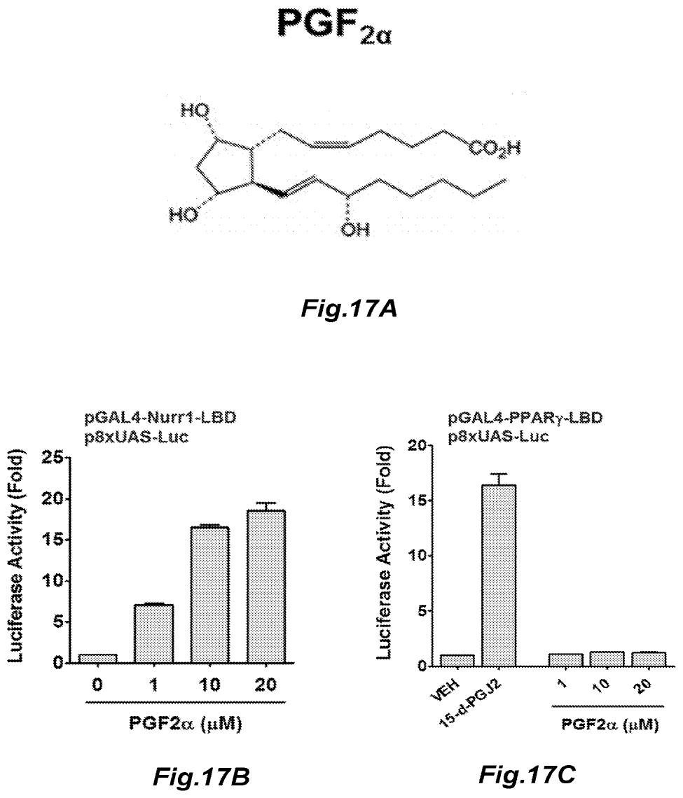

Nurr1-LBD and PPARγ-LBD transcriptional activity was responsive to a number of cyclopentenone PGs (FIG. 17). PGEs and PGHs activate transcriptional function of Nurr1 but not that of PPARγ (FIG. 16A-16C). PGF2a activates transcriptional function of Nurr1 but not that of PPARγ (FIG. 17A-17B).

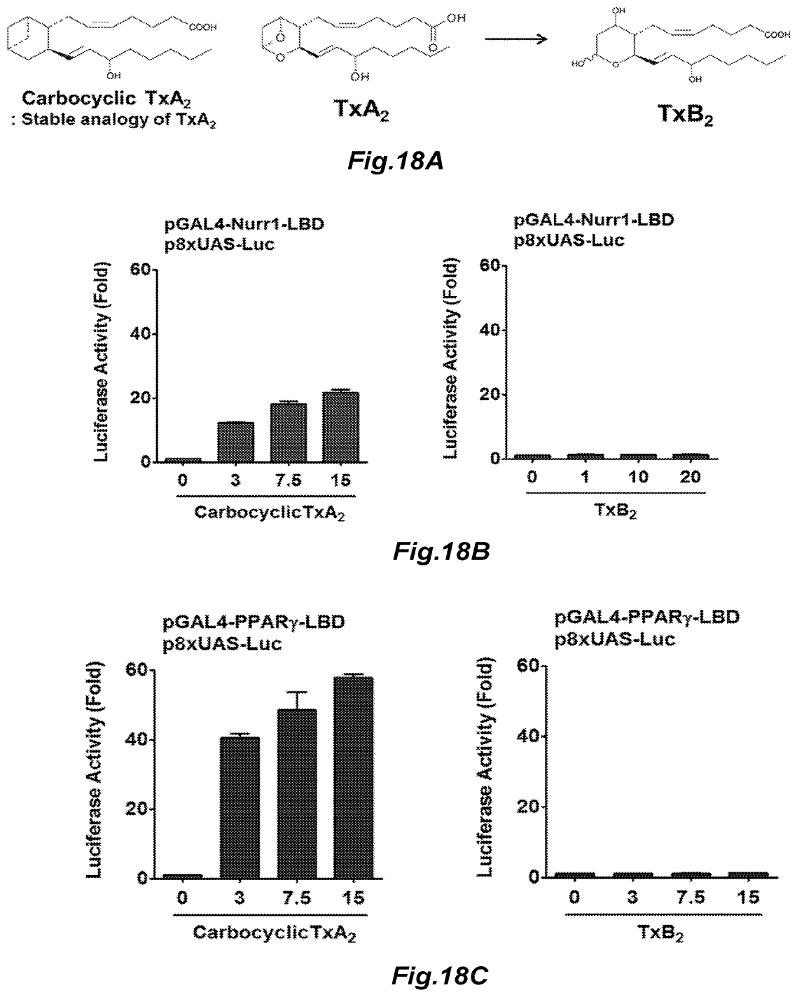

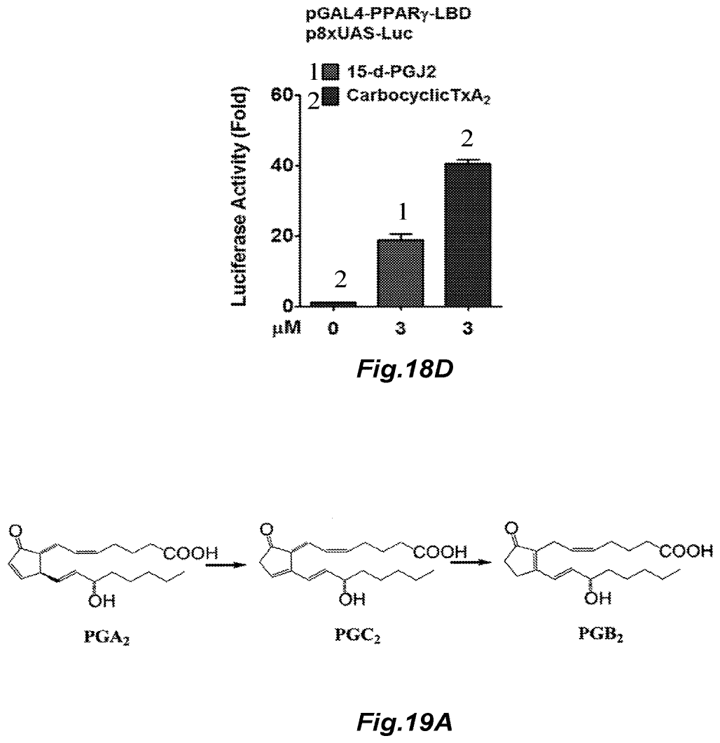

Carbocyclic thromboxane A2 (TxA2) activates transcriptional function of Nurr1 and PPARγ in a dose-dependent manner (FIG. 18A-18D). In contrast, TxB2 does not activate transcriptional function of Nurr1 and PPARγ.

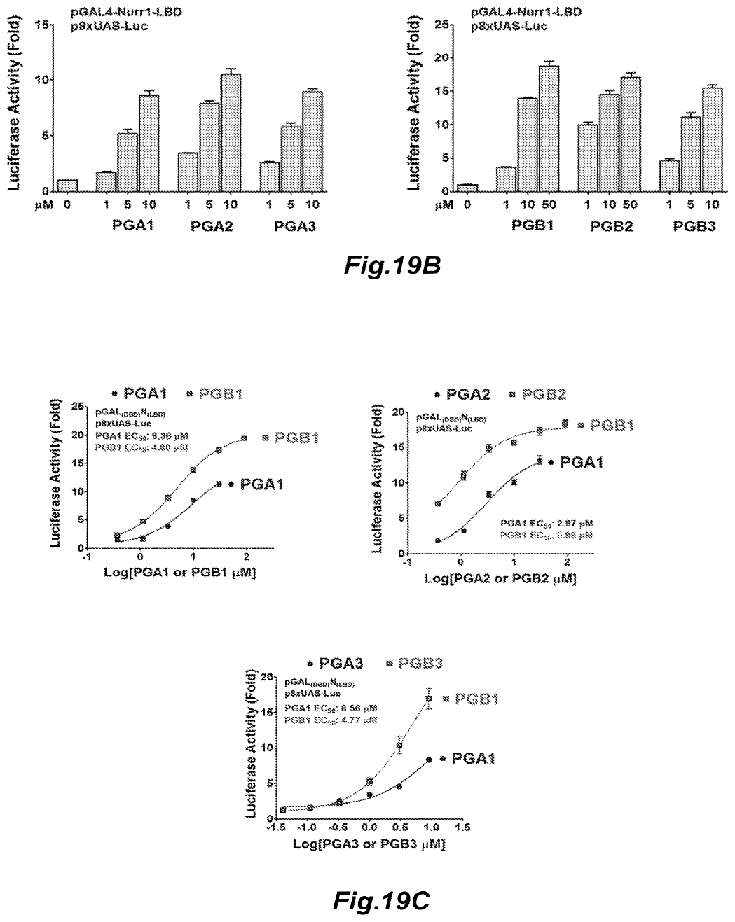

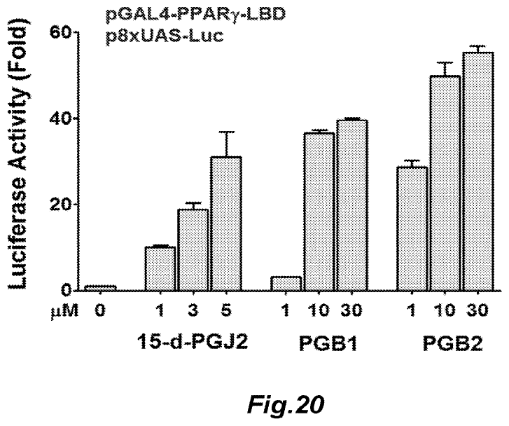

PGAs and PGBs activate transcriptional function of Nurr1 in a dose-dependent manner (FIG. 19A-19C). PGB1 and PGB2 activate transcriptional function of PPARγ in a dose-dependent manner (FIG. 20).

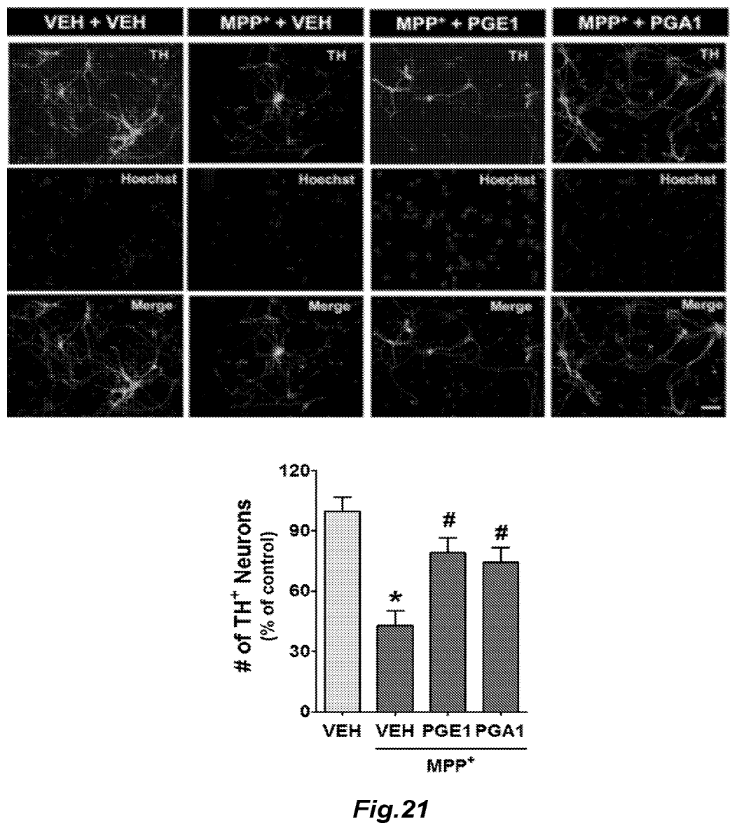

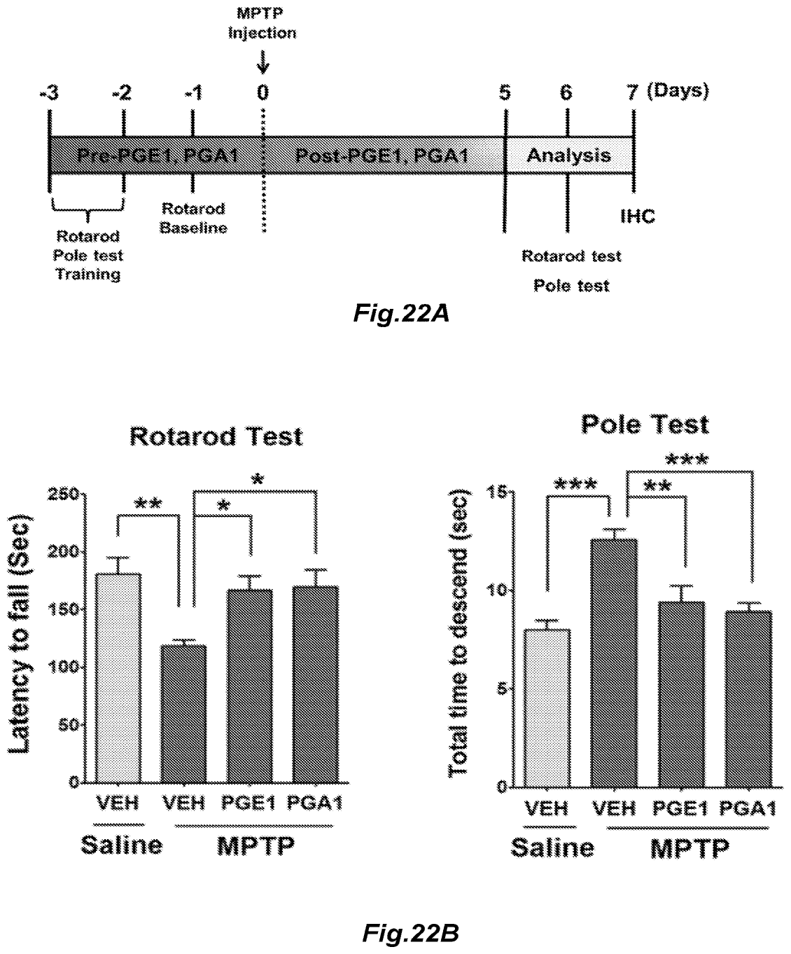

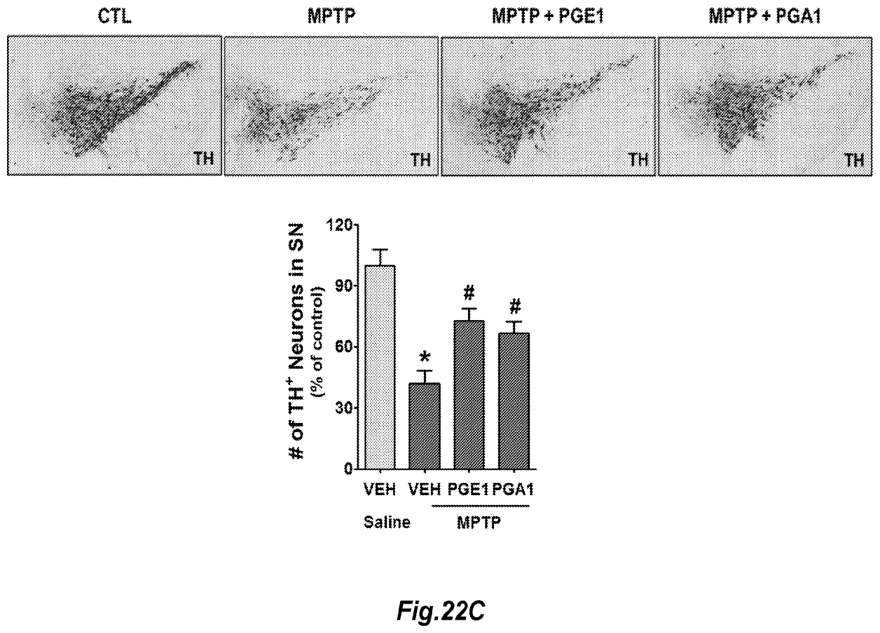

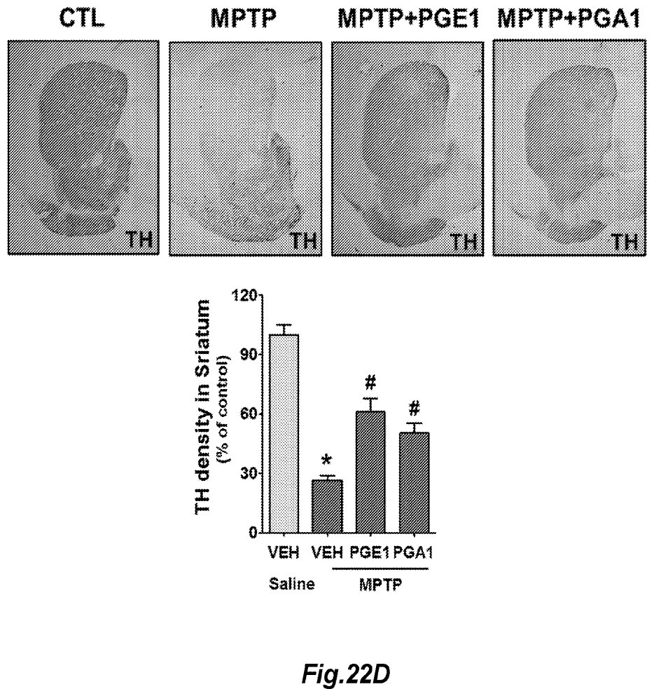

PGE and PGA have a protective effect on primary cultured mDA neurons (FIG. 21) and in a MPTP-induced animal model of PD (FIG. 22A-22D).

### Example 4

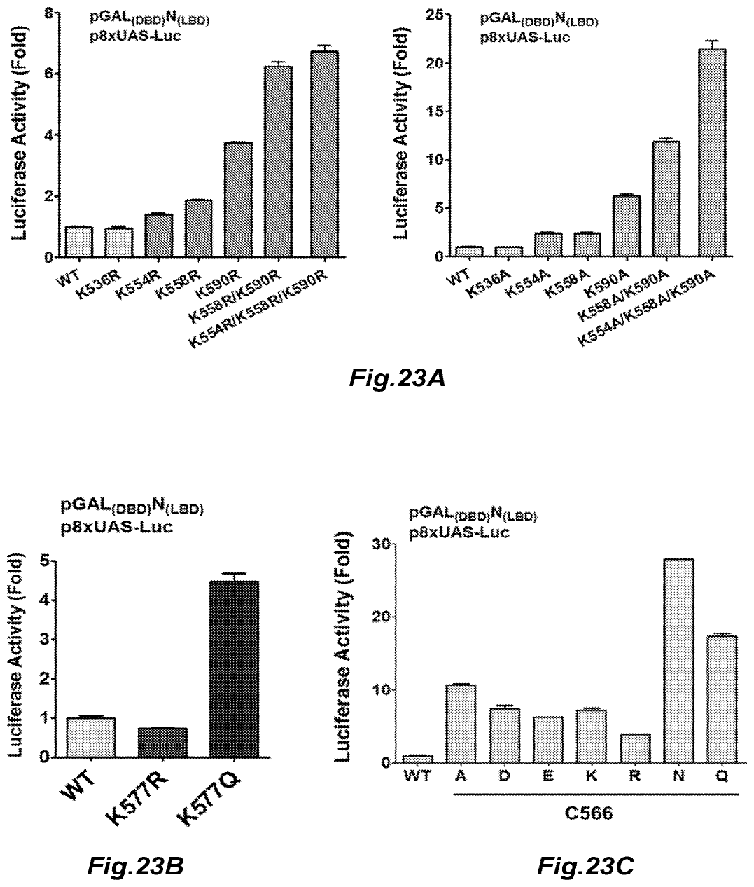

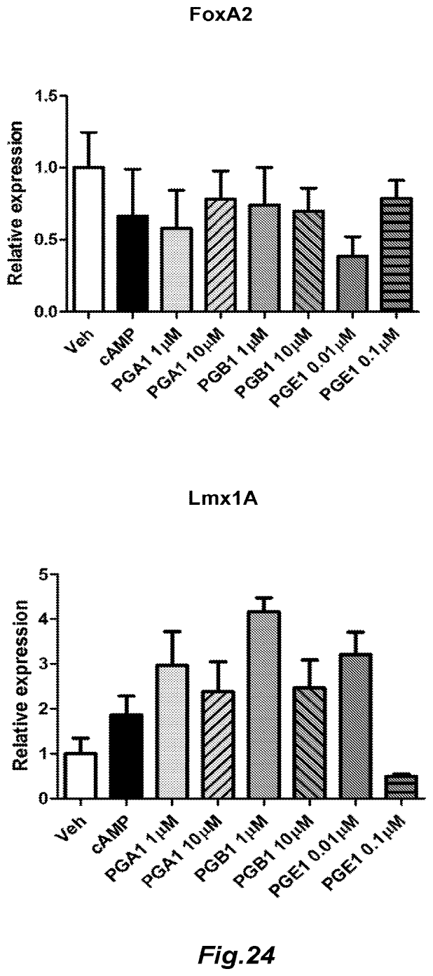

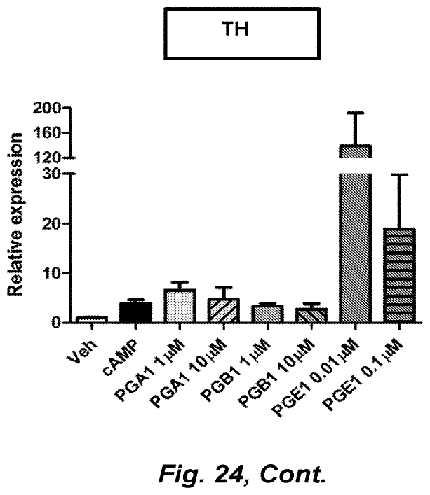

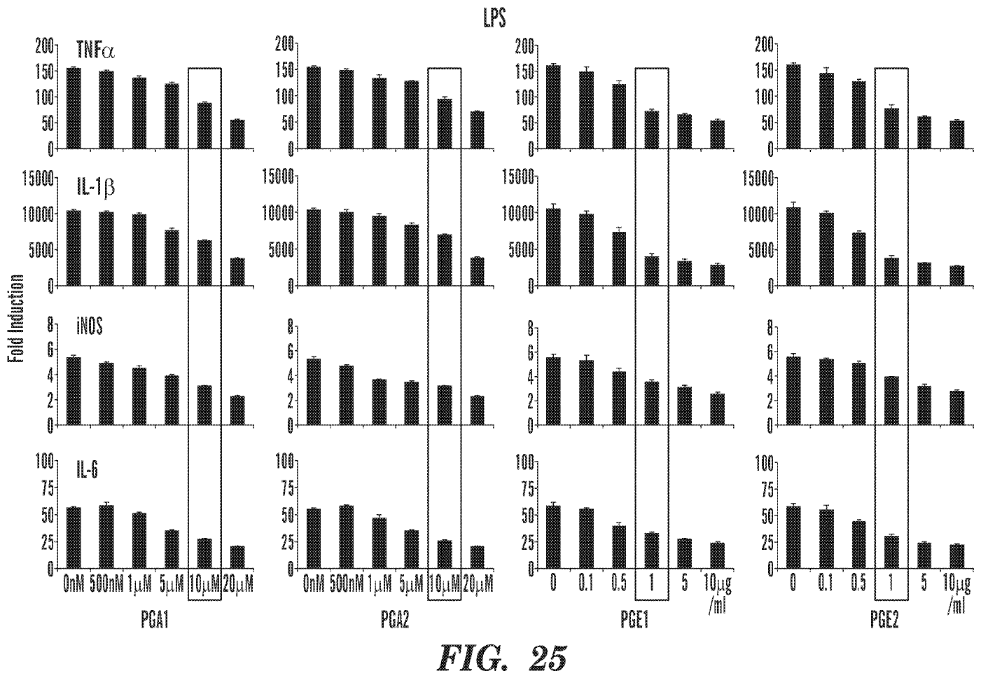

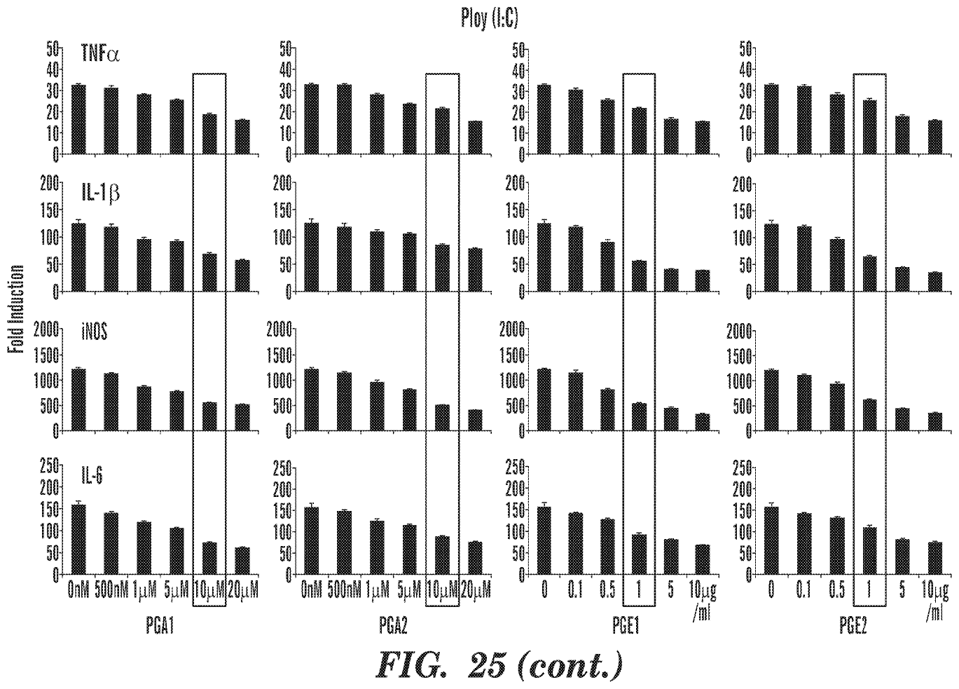

Mutational analysis of Nurr1-LBD revealed that single mutations at K554, K558, or K590 residues of Nurr1-LBD into arginine (R) residue show a potentiated basal transcriptional activity comparing to wild-type Nurr1-LBD. Moreover, double (K558R/K590R) or triple (K554R/K558R/K590R) mutations exhibit a synergistic potentiation in Nurr1 basal activity comparing to wild-type Nurr1 (FIG. 23A). Acetylated mimic form (K577Q) of Nurr1-LBD also shows a potentiated basal transcriptional activity comparing to wild-type Nurr1-LBD (FIG. 23B). FIG. 23C. In the C566 residue on Nurr1-LBD, point mutations on C566 residue into several residues (A, D, E, K, R, N, Q) also exhibit a potentiated transcriptional activity of Nurr1 comparing to wild-type Nurr1 (FIG. 23C).

Nurrl polypeptideNCBI Ref Seq: NP_001132981.1
SEQ ID NO: 1

| | |
|---|---|
| 1 | mpcvqaqygs spqgaspasq sysyhssgey |
| | ssdfltpefv kfsmdltnte itattslpsf |
| 61 | stfmdnystg ydvkppclyq mplsgqqssi |
| | kvediqmhny qqhshlppqs eemmphsgsv |
| 121 | yykpsspptp stpsfqvqhs pmwddpgslh |
| | nfhqnyvatt hmieqrktpv srlslfsfkq |
| 181 | sppgtpvssc qmrfdgplhv pmnpepagsh |
| | hvvdgqtfav pnpirkpasm gfpglqigha |
| 241 | sqlldtqvps ppsrgspsne glcavcgdna |
| | acqhygvrtc egckgffkrt vqknakyvcl |
| 301 | ankncpvdkr rrnrcqycrf qkclavgmvk |
| | evvrtdslkg rrgripskpk spqdpsppsp |
| 361 | pvslisalvr ahvdsnpamt sldysrfqan |
| | pdyqmsgddt qhiqqfydll tgsmeiirgw |
| 421 | aekipgfadl pkadqdllfe saflelfvlr |
| | layrsnpveg klifcngvvl hrlqcvrgfg |
| 481 | ewidsivefs snlqnmnidi safsciaala |
| | mvterhglke pkrveelqnk ivnclkdhvt |
| 541 | fnngglnrpn ylskllgklp elrtlctqgl |
| | qrifylkled Ivpppaiidk Ifldtlpf |

Nurrl polypeptideNCBI Ref Seq: NP_038641.1
SEQ ID NO: 2

| | |
|---|---|
| 1 | mpcvqaqygs spqgaspasq sysyhssgey |
| | ssdfltpefv kfsmdltnte itattslpsf |
| 61 | stfmdnystg ydvkppclyq mplsgqqssi |
| | kvediqmhny qqhshlppqs eemmphsgsv |
| 121 | yykpsspptp stpsfqvqhs pmwddpgslh |
| | nfhqnyvatt hmieqrktpv srlslfsfkq |
| 181 | sppgtpvssc qmrfdgplhv pmnpepagsh |
| | hvvdgqtfav pnpirkpasm gfpglqigha |
| 241 | sqlldtqvps ppsrgspsne glcavcgdna |
| | acqhygvrtc egckgffkrt vqknakyvcl |
| 301 | ankncpvdkr rrnrcqycrf qkclavgmvk |
| | evvrtdslkg rrgrlpskpk spqdpsppsp |
| 361 | pvslisalvr ahvdsnpamt sidysrfqan |
| | pdyqmsgddt qhiqqfydll tgsmeiirgw |
| 421 | aekipgfadl pkadqdllfe saflelfvir |
| | layrsnpveg klifcngvvl hrlqcvrgfg |
| 481 | ewidsivefs snlqnmnidi safsciaala |
| | mvterhglke pkrveelqnk ivnclkdhvt |
| 541 | fnngglnrpn ylskllgklp elrtlctqgl |
| | qrifylkled ivpppaiidk ifldtlpf |

1. A method of treating Parkinson's disease in a subject in need thereof, the method comprising administering to the subject at least one of:
prostaglandin E (PGE) 1; PGE2: PGE3; prostaglandin H(PGH)1; PGH2; PGH3; prostaglandin F (PFG)2a; prostaglandin A (PGA)1; PGA2; PGA3; prostaglandin B (PGB)1; PGB2; PGB3; PGJ2; 15-d-PGJ2; and/or carbocyclic TxA2.

2. (canceled)

3. The method of claim 1, wherein the subject is further administered chloroquine or a choloroquine derivative.

4. The method of claim 1, wherein the subject is administered at least one of:
PGB1; PGB2; PGB3; PGJ2; 15-d-PGJ2; and carbocyclic TxA2.

5. The method of claim 1, wherein the subject is administered at least one of:
PGB1; PGB2; or PGB3.

6. The method of claim 1, wherein the subject is not administered PGE3.

7. The method of claim 1, wherein the subject is not administered PGE1; PGE2; or PGE3.

8. A method of increasing the level and/or activity of Nurr1 or PPARγ in a cell, the method comprising contacting the cell with at least one of:
PGE1; PGE2: PGE3; PGH1; PGH2; PGH3; PGF2a; PGA1; PGA2; PGA3; PGB1;
PGB2; PGB3; PGJ2; 15-d-PGJ2; and/or carbocyclic TxA2

9. The method of claim 8, wherein the level of Nurr1 is the level of Nurr1 mRNA transcript.

10. A method of treating a Nurr1-mediated and/or PPARγ-mediated condition in a subject in need thereof, the method comprising administering to the subject a vector comprising a nucleic acid sequence encoding a Nurr1 polypeptide.

11. The method of claim 10, wherein the Nurr1 polypeptide comprises an amino acid substitution at one or more residues corresponding to K554, K558, K590, K577, and C566 or SEQ ID NO; 1 or 2.

12. The method of claim 10, wherein the Nurr1 polypeptide encoded by the vector is transcribed in the subject at the same or higher transcriptional level than the endogenous Nurr1.

13. The method of claim 10, wherein the Nurr1-mediated and/or PPARγ-mediated condition is selected from the group consisting of:
neurodegenerative disorders; an inflammatory disease; Parkinson's disease; Alzheimer's disease; Schizophrenia; immune disorders; mild cognitive impairment; restless leg syndrome; and autoimmune disorders.

14.-33. (canceled)
